(19) United States
(12) Patent Application Publication
(10) Pub. No.: US 2003/0039964 A1
(43) Pub. Date: Feb. 27, 2003

(54) Method

(76) Inventors: Philip Morrison Giffard, Queensland (AU); Charles P. Morris, Queensland (AU); Joanne Voisey, Queensland (AU); Gregory Coia, Victoria (AU); David Scott Hammond, Queensland (AU); Veronica Brockhurst, Queensland (AU)

Correspondence Address: Robert M. O'Keefe, O'KEEFE, EGAN & PETERMAN, Building C, Suite 200, 1101 Capital of Texas Highway South, Austin, TX, 78746, US

(21) Appl. No.: 09/852,058

(22) Filed: May 9, 2001

Related U.S. Application Data
(60) Provisional application No. 60/202,797, filed on May 9, 2000.

(30) Foreign Application Priority Data
May 9, 2001 (AU) PCT/AU01/00527

(51) Int. Cl. C12Q 1/68; C07H 21/04

(52) U.S. Cl. 435/6; 536/24.3

(57) ABSTRACT

The present invention relates generally to a method for detecting a nucleic acid molecule having a particular nucleotide sequence. Such a nucleic acid molecule is generally referred to as the “target” sequence or molecule. The method of the present invention generally comprises the use of competitive priming of pre- or post-amplified nucleic acid molecules. The nucleic acid molecules subjected to such primer interrogation are generally immobilized to a solid support by hybridization of a target molecule to a primer anchored to a solid phase. Amplimer-mediated bridging of a particular primer, -labelled or unlabelled, is then used to detect the presence of a primer having a selected sequence. The method of the present invention is useful in a range of applications including inter alia diagnosis, nucleotide sequencing and the screening for nucleic acid-modifying molecules such as carcinogens. The instant method may also be used to discriminate between nucleotide repeat number polymorphism including microsatellite repeat alleles occurring in a range of neurodegenerative and other disease conditions including Huntington's disease which result from or have a causative nature associated with repeat expansion. The subject method may also be used to quantitate target nucleic acid molecules. The present invention further combines the subject methodology and microchip technology to permit interrogation of target sequences in a high through-put manner.

### FIELD OF THE INVENTION

The present invention relates generally to a method for detecting a nucleic acid molecule having a particular nucleotide sequence. Such a nucleic acid molecule is generally referred to as the “target” sequence or molecule. The method of the present invention generally comprises the use of competitive priming of pre- or post-amplified nucleic acid molecules. The nucleic acid molecules subjected to such primer interrogation are generally immobilized to a solid support by hybridization of a target molecule to a primer anchored to a solid phase. Amplimer-mediated bridging of a particular primer, labelled or unlabelled, is then used to detect the presence of a primer having a selected sequence. The method of the present invention is useful in a range of applications including inter alia diagnosis, nucleotide sequencing and the screening for nucleic acid-modifying molecules such as carcinogens. The instant method may also be used to discriminate between nucleotide repeat number polymorphism including microsatellite repeat alleles occurring in a range of neurodegenerative and other disease conditions including Huntington's disease which result from or have a causative nature associated with repeat expansion. The subject method may also be used to quantitate target nucleic acid molecules. The present invention further combines the subject methodology and microchip technology to permit interrogation of target sequences in a high through-put manner.

### BACKGROUND OF THE INVENTION

Bibliographic details of the publications numerically referred to in this specification are collected at the end of the description.

Reference to any prior art in this specification is not, and should not be taken as, an acknowledgment or any form of suggestion that this prior art forms part of the common general knowledge in Australia or any other country.

The increasing sophistication of molecular biological techniques is greatly facilitating research and development in the medical and agricultural industries. Of particular importance is the ability to detect specific nucleotide sequences. Generally, detection requires target nucleic acid molecules to be amplified.

A variety of nucleic acid amplification techniques exist for the diagnosis of infectious and genetic diseases. Since its development over a decade ago, the polymerase chain reaction [PCR] (1) has become the method of choice in research and DNA-based diagnostics. This can be attributed to its speed and relative simplicity. Other amplification techniques include the ligase chain reaction [LCR] (2) and the transcription-based amplification system [TAS] (3).

Various other amplification techniques exist which do not require extensive thermal cycling and are essentially isothermal systems. Several of these are transcription-mediated or require RNA as an integral component of the reaction therefore necessitating that the amplification environment is kept free from ribonuclease contamination. These methods include the Qβ replicase system (4), self-sustained sequence replication [3SR] (5) and nucleic acid sequence-based amplification [NASBA] (6).

Presently, there appear to exist at least two isothermal techniques for the amplification of nucleic acid sequences which essentially do not require RNA intermediates. Strand displacement amplification [SDA] (7) is an isothermal technique which relies on the ability of a restriction enzyme to nick a hemiphosphorothioated recognition site and the ability of a polymerase to initiate replication at a nick and displace the downstream stand. The other isothermal technique which can be used to amplify a nucleic acid sequence is rolling circle amplification (RCA).

Various forms of a rolling circle amplification technique have previously been described (8, 9). In essence, the technique relies on amplification from a circular DNA probe. The circular probe, commonly referred to as a “padlock probe”, is designed such that it has regions at both its 5′ and 3′ ends which are complementary to the target sequence of interest and are separated by a region of nucleotides of non-target derived origin. Upon hybridization, the 5′ and 3′ ends of the probe are brought into close proximity to one another. If the two probe regions are adjacent to one another the 5′ and 3′ ends can be joined to produce a circular probe. In some instances, however, the probe regions are separated from one another by a small stretch of nucleotides. This region must be filled to achieve the generation of a circular probe. In this regard, a various of techniques can be utilized including the use of spacer oligonucleotides or by using a DNA polymerase (or a reverse transcriptase in the case of an RNA target) in combination which deoxynucleotide triphosphate molecules to fill the gap prior to ligation.

Hybridization is extensively used to quantitatively or qualitatively identify nucleic acid molecules. Hybridization occurs when there is sufficient complementarity between two nucleotide sequences to effect non-covalent binding between the two sequences. Generally, labelled nucleotide acid primers or probes are used to hybridize to a target sequence. In addition, or alternatively, labelled nucleic acid molecules are incorporated into target nucleic acid molecules during or following amplification.

The amplification and identification of nucleic acid molecules may also be combined with solid phase procedures. Solid phase amplification (SPA) is described, for example, in International Patent Application No. PCT/AU92/00587 [WO 93/09250; published May 13, 1993]. In SPA, amplification occurs in the presence of both a first, immobilized primer and a second labelled primer which hybridizes to a target nucleic acid in the opposite direction to the first.

Despite the usefulness and widespread applicability of amplification and hybridization procedures, there is a need to improve the sensitivity and efficacy of nucleic acid detection procedures.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers SEQ ID NO:1, SEQ ID NO:2, etc. A sequence listing is provided after the claims.

One aspect of the present invention contemplates a method for detecting a nucleic acid molecule having a particular nucleotide sequence immobilized to a solid support via hybridization of said nucleic acid molecule to a primer anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is for a time and under conditions sufficient for the nucleic acid primer which is complementary to the target sequence to hybridize to said target sequence with greater efficiency and/or specificity compared to the nucleic acid primer which contains a mis-match and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

Another aspect of the present invention provides a method for detecting a nucleic acid molecule having a particular nucleotide sequence immobilized to a solid support via hybridization of said nucleic acid molecule to a primer anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

Yet another aspect of the present invention provides a method for detecting a nucleic acid molecule having a particular nucleotide sequence anchored to a solid support via hybridization of said nucleic acid molecule to a primer anchored to said solid support, said method comprising subjecting said nucleic acid molecule to amplification using at least two solution phase primers and anchoring said amplified nucleic acid molecule to the solid support, contacting said anchored nucleic acid molecule with at least two other solution phase nucleic acid primers wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence with greater efficiency and/or specificity compared to the nucleic acid primer which contains a mis-match and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

Still yet another aspect of the present invention contemplates a method for detecting a nucleic acid molecule having a particular nucleotide sequence anchored to a solid support via hybridization of said nucleic acid molecule to a primer anchored to said solid support, said method comprising subjecting said nucleic acid molecule to amplification using at least two solution phase primers and anchoring said amplified nucleic acid molecule to the solid support, contacting said anchored nucleic acid molecule with at least two other solution phase nucleic acid primers wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

Even yet another aspect of the present invention provides a solid support comprising an array of immobilized primers wherein each of the primers may comprise the identical nucleotide sequence or one or more may differ from each other by at least one nucleotide and wherein the array is used to detect a nucleic acid molecule having a particular nucleotide sequence immobilized to a solid support via hybridization of said nucleic acid molecule to said one or more primers anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence with greater efficiency and/or specificity compared to the nucleic acid primer which contains a mis-match and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

Still yet another aspect of the present invention provides a solid support comprising an array of immobilized primers wherein each of the primers may comprise the identical nucleotide sequence or one or more may differ from each other by at least one nucleotide and wherein the array is used to detect a nucleic acid molecule having a particular nucleotide sequence immobilized to a solid support via hybridization of said nucleic acid molecule to said one or more primers anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence but at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

Another aspect of the present invention contemplates a method for detecting a nucleic acid molecule having a particular nucleotide sequence anchored to a solid support via hybridization of said nucleic acid molecule to a primer anchored to said solid support, said method comprising subjecting said nucleic acid molecule to amplification using at least two solution phase primers having a high Tm and wherein the amplification conditions are such that said solution phase primers are active and anchoring said amplified nucleic acid molecule to the solid support, contacting said anchored nucleic acid molecule with at least two other solution phase nucleic acid primers wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said latter two primers have a Tₘ lower than the temperature used in said first amplification such that during the first amplification, the second set of primers is inactive but during said second amplification, said second set of primers is active, wherein said contact is under conditions sufficient for the nucleic acid primer which is complementary to the target sequence to hybridize to said target sequence greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

Yet another aspect present invention contemplates a method for detecting a nucleic acid molecule having a particular nucleotide sequence anchored to a solid support via hybridization of said nucleic acid molecule to a primer anchored to said solid support, said method comprising subjecting said nucleic acid molecule to amplification using a solution phase primer having a high Tₘ and wherein the amplification conditions are such that said solution phase primers are active and anchoring said amplified nucleic acid molecule to the solid support, contacting said anchored nucleic acid molecule with at least two other solution phase nucleic acid primers each having a low Tₘ wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said latter two primers have a Tₘ lower than the temperature used in said first amplification such that during the first amplification, the second set of primers is inactive but during said second amplification, said second set of primers is active, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

Still another aspect of the present invention contemplates a method for quantitating a nucleic acid molecule immobilized to a solid support via hybridization of said nucleic acid molecule to a primer anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least to primers is labelled with a reporter molecule capable of providing an identifiable signal, wherein said contact is under conditions sufficient for the nucleic acid primers to amplify said target sequence to completion of an amplification reaction wherein the amount of target nucleic acid molecule is proportional to the number of amplification cycles such that the amount of target nucleic acid molecule is determined from the ratio of incorporation of complementary and mis-match primers at the end of the amplification reaction.

Still yet another aspect contemplates a method for detecting a nucleotide length polymorphism in a target nucleic acid molecule, said method comprising subjecting said target nucleic acid molecule to interrogation by a pair of immobilized primers wherein said primers are immobilized in separate reaction vessels or separate spot arrays and wherein one primer is capable of priming a particular nucleotide length polymorphism whereas the other primer is unable to induce priming of said nucleotide length polymorphism wherein at least one primer is labelled with a reporter molecule capable of providing an identifiable signal wherein the relative presence or absence of said signal is indicative of the presence or absence of said nucleotide length polymorphismn.

### BRIEF DESCRIPTION OF THE FIGURES

FIG. 1 is a diagrammatic representation of the essential elements of solid phase cascade rolling circle amplification (CRCA). Target and ligase mediated padlock circularization precedes CRCA with one immobilised primer and one or more labelled solution phase primers. During the exponential phase of the reaction, it is likely that the extended labelled primer is displaced by subsequent priming events and then serves as a template and/or primer for additional extension reactions, thus yielding a complex network of linear double stranded concatemers. Therefore, the labelled primer may frequently prime on a linear molecule that is not directly connected to the circular template.

FIG. 2 is a representation of solid phase CRCA (SPCRCA) using synthetic Chlamydia derived targets, padlock, spacers and primers. The ligation reactions contained 50 pmol target unless otherwise indicated. The concentrations of other reagents were as stated in Example 1. (A) Absorbance readings after 60 min colour development. 1. Negative control 1, no target, C. pneunoniae-specific spacer and primer; 2. negative control 2, C. pneumoniae-specific target, spacer and primer, no ligase; 3. C. pneumoniae-specific target, spacer and primer; 4. C. pneumoniae-specific target, C. trachomatis specific spacer and primer; 5. C. trachomatis-specific target, spacer and primer; 6. C. trachomatis specific target, C. pneumoniae-specific spacer and primer; 7. Negative control 3, colour development regents only. (B) Electrophoresis in 2% w/v agarose of the solution phase material from A. Lane M. molecular weight marker (base pairs indicated). Lanes 1-7 as for A.

FIG. 3 is a representation of post amplification competitively primed SPCRCA using Factor V Leiden mutation derived target, padlock, spacers and primers. 1 pmol synthetic target was used. The first non-specific amplification was carried out in microfuge tubes. (A) Absorbance readings after 30 min colour development. 1. No target, second amplification reaction only, mutant specific spacer, labelled mutant-specific primer, unlabelled WT-specific primer; 2. As for 1 but both amplification reactions were carried out; 3. WT-specific target and spacer for first amplification, labelled WT-specific primer and unlabelled mutant specific primers in second amplification; 4. WT target and spacer for first amplification, labelled mutant specific and unlabelled WT specific primers in second amplification; 5. mutant-specific target and spacer in first amplification, labelled mutant specific and unlabelled WT specific primers in second amplification; 6. mutant-specific target and spacer in first amplification, labelled WT-specific primer and unlabelled mutant specific primers in second amplification; 7-8. colour development reagents only. (B) Electrophoresis of the solution phase material from A. M. Molecular weight markers (base pairs indicated). 1-6 as for A.

FIG. 4 is a representation of post-amplification competitively primed SPCRCA using Factor V Leiden mutation derived target, padlock, spacers and primers. 500 ng of human genomic DNA was used as the target. The first and second amplification reactions were carried out in the same micro-wells. (A) Absorbance readings after 30 min colour development. 1. No target, mutant specific spacer, labelled mutant specific primer, unlabelled WT-specific primer. Both amplification reactions were carried out; 2. homozygous WT target, WT spacer, labelled WT-specific primer, unlabelled mutant-specific primer; 3. homozygous WT-specific target, WT-specific spacer, labelled mutant-specific primer, unlabelled WT-specific primer; 4. heterozygous mutant target, mutant-specific spacer, labelled mutant-specific primer, unlabelled WT-specific primer; 5. heterozygous mutant target, mutant-specific spacer, labelled WT-specific primer, unlabelled mutant-specific primer. (B) Electrophoresis of the solution phase material from A. M. Molecular weight markers (base pairs indicated). 1-5 as for A.

FIG. 5 is a diagrammatic representation of the instant method discriminating between microsatellite repeat number alleles.

FIG. 6 is a diagrammatic representation showing competitive priming with differential capture.

FIG. 7 is a photographic representation showing 10 cycle, 10 ng template, AS-PCR for uidA β-glucuronidase SNP.

FIG. 8 is a photographic representation showing 10 cycle, 10 ng template, COP-PCR for uidA β-glucuronidase SNP.

FIG. 9 is a diagrammatic representation showing non-competitive hybridization to detect repeat length polymorphism in a target DNA molecule. The repeat region is indicated by wavy line. In this embodiment, the non-competitive hybridization is conducted by two immobilized primers where one primer is capable of inducing priming and incorporation of a label whereas the other primer, due to its length, is not capable of either function. The presence or absence of a detectable signal from the label indicates the presence or absence of the repeat length polymorphism.

### SUMMARY OF DNA OLIGONUCLEOTIDES

| Primer description | Sequence identifier |
|---|---|
| SPACERW | SEQ ID NO:1^{1,2} |
| SPACERM | SEQ ID NO:2^{1,2} |
| PADLOCK | SEQ ID NO:3² |
| FVA | SEQ ID NO:4 |
| FVComT2 | SEQ ID NO:5² |
| FVW2 | SEQ ID NO:6³ |
| FVM2 | SEQ ID NO:7³ |
| FV5 | SEQ ID NO:8 |
| FV6 | SEQ ID NO:9 |
| NATF | SEQ ID NO:10 |
| NATR | SEQ ID NO:11 |
| Chlamydia—specific padlock | SEQ ID NO:12 |
| Chlamydia pneumoniae—derived target | SEQ ID NO:13 |
| Chlamydia pneumoniae—specific spacer | SEQ ID NO:14 |
| Chlamydia trachomatis—derived target | SEQ ID NO:15 |
| Chlamydia trachomatis—specific spacer | SEQ ID NO:16 |
| Chlamydia pneumonia—specific | SEQ ID NO:17 |
| amplification primer | |
| Chlamydia trachomatis—specific | SEQ ID NO:18 |
| amplification primer | |
| Wild-type FVComT sequence | SEQ ID NO:19 |
| Mutant FVComT sequence | SEQ ID NO:20 |
| UidA3′ outer | SEQ ID NO:21 |
| UidASPA3′FAM | SEQ ID NO:22 |
| UidAsm0157-B | SEQ ID NO:23 |
| UidAsm1O157 | SEQ ID NO:24 |
| UidAsmEco-B | SEQ ID NO:25 |

¹Nucleotide T at position 9 is biotinylated
²Nucleotide C at position 1 is phosphorylated
³Nucleotide C at position 1 is FAM

### DETAILED DESCRIPTION OF THE PREFERRED EMBODIMENTS

The present invention provides an efficacious method for detecting a nucleic acid molecule having a particular nucleotide sequence. The method is based on a combination of solid phase amplification (SPA) and competitive priming. The method is particularly applicable for detecting nucleic acid molecules exhibiting a single nucleotide specificity of interest such as a polymorphic or allelic forms of a gene. One particular form of the instant method is referred to herein as solid phase cascade rolling circle amplification (SPCRCA). Reference herein to a “nucleic acid molecule” or “target nucleic acid molecule” includes reference to DNA (e.g. cDNA or genomic DNA) or RNA (e.g. mRNA).

Accordingly, one aspect of the present invention contemplates a method for detecting a nucleic acid molecule having a particular nucleotide sequence immobilized to a solid support via hybridization of said nucleic acid molecule to a primer anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is for a time and under conditions sufficient for the nucleic acid primer which is complementary to the target sequence to hybridize to said target sequence with greater efficiency and/or specificity compared to the nucleic acid primer which contains a mis-match and then detecting the relative presence of a signal wherein the relative presence of said signal is indicative of which primer has hybridized to the target sequence depending on which primer hasp been labelled and the relative presence of a signal is indicative of the identity of the immobilized nucleic acid molecule.

Reference herein to a “primer” is not to be taken as any limitation as to structure, size or function. The primer may be used as an amplification molecule or may be used as a probe for hybridization purposes. The preferred form of the molecule is as a primer for amplification.

Reference herein to a “nucleic acid primer” includes reference to a sequence of deoxyribonucleotides or ribonucleotides comprising at least 3 nucleotides. Generally, the nucleic acid primer comprises from about 3 to about 100 nucleotides, preferably from about 5 to about 50 nucleotides and even more preferably from about 5 to about 25 nucleotides. A primer having less than 50 nucleotides may also be referred to herein as an “oligonucleotide primer”. The primers of the present invention may be synthetically produced by, for example, the stepwise addition of nucleotides or may be fragments, parts, portions or extension products of other nucleotide acid molecules. The term “primer” is used in its most general sense to include any length of nucleotides which, when used for amplification purposes, can provide a free 3′ hydroxyl group for the initiation of DNA synthesis by a DNA polymerase. DNA synthesis results in the extension of the primer to produce a primer extension product complementary to the nucleic acid strand to which the primer has hybridized. Importantly, with respect to the primers, differential washing is employed to remove unextended primers which could anneal to an immobilized amplimer. In essence, unextended primers are removed such as by washing at a temperature that melts a primer-single-stranded amplimer complexes but does not substantially disrupt fully double-stranded amplimers.

Reference to greater efficiency or specificity includes reference to a greater likelihood of hybridization in a complementary primer compared to a mis-matched primer. Conveniently, efficiency and/or specificity can be measured following post-amplification primer interrogation where the complementary primer allows significant extension compared to a mis-matched primer.

Reference to “relative presence” includes reference to “relative absence”.

In a preferred embodiment, one of the at least two solution phase nucleic acid primers is involved in an amplification reaction to amplify a target sequence. If this primer is also labelled with a reporter molecule, the amplification reaction will result in incorporation of the label in the amplified product. The terms “amplification product” and “amplimer” may be used interchangably.

Accordingly, another aspect of the present invention provides a method for detecting a nucleic acid molecule having a particular nucleotide sequence immobilized to a solid support via hybridization of said nucleic acid molecule to a primer anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence and to extend from its 3′ terminus to form an extension product complementary to the stand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

The extension of the hybridized primer to produce an extension product is included herein by the term “amplification”. Amplification generally occurs in cycles of denaturation followed by primer hybridization and extension. The present invention encompasses from about 1 cycle to about 120 cycles, preferably from about 2 to about 70 cycles and even more preferably from about 5 to about 40 cycles including about 10, 15, 20, 25 and 30 cycles.

The nucleic acid primers having single or multiple base differences are also referred to herein as “competitive primers”. Generally, the competitive primers are nucleic acid molecules of the same length but differ by at least one base mis-match. In one aspect, by manipulating hybridization and stringency conditions, the primers compete with each other for the ability to hybridize to the target sequence. Under particular stringency conditions, only the primer having the most complementarity to the target sequence will hybridize. The primer with the least one complementarity will, under these conditions, substantially not hybridize. More particularly, however, the difference between a complementary and mis-matched primer is determined by the efficiency and/or specificity of elongation. Accordingly, complementary primers will elongate more preferentially relative to mis-matched primers. In this aspect, the conditions are manipulated to induced preferential extension of the 3′ terminus of the primer. As stated above, unextended primers are washed away at temperatures which melt primer-single-stranded amplimer complexes but which does not disrupt fully double-stranded amplimers.

A base mis-match occurs when two nucleotide sequence are aligned with substantial complementarity but at least one base aligns to a base which would result in an “abnormal” binding pair. An abnormal binding pair occurs if thymine (T) were to bind to a base other than adenine (A), if A were to bind to a base other than T, if guanine (G) were to bind to a base other than cytosine (C) or if C were to bind to a base other than G.

In accordance with the present invention, primers are selected with nucleotide sequences substantially complementary to a particular target sequence. For example, the primers may be selected to identify a polymorphism. A polymorphism is a nucleotide variation at a genetic locus. Certain disease conditions, for example, can be attributed to or happen to coincide with one particular base sequence but not another. This difference is a genetic polymorphism. A polymorphism resulting in a disease condition is also referred to as a mutated genetic sequence.

In accordance with the present invention, a sample of nucleic acid to be tested is added to a chamber, well or other receptacle comprising an immobilized nucleic acid capture molecule. The capture molecules comprises a nucleotide sequence substantially complementary to a portion of either the target nucleotide sequence or a nucleotide sequence within a nucleic acid molecule comprising the target sequence. The terms “captive molecule” and “primer” may be used interchangably.

The capture molecule may be immobilized to the solid phase by any convenient means. The solid phase may be any structure having a surface which can be derivatized to anchor a nucleic acid primer or other capture molecule. Preferably, the solid phase is a planar material such as the side of a microtiter well or the side of a dipstick.

The anchored nucleic acid molecule generally needs to be able to capture a target nucleic acid molecule by hybridization and optionally participate in an amplification reaction. Alternatively, the anchored nucleic acid molecule will capture amplified nucleic acid molecules. The former, however, is preferred.

Methods for linking nucleic acid molecules to solid supports are well known in the art. Processes for linking the primer to the solid phase include amide linkage, amidate linkage, thioether linkage and the introduction of amino groups on to the solid phase. Examples of linkage to a solid phase can be found in International Patent Application No. PCT/AU92/00587 [WO 93/09250].

The anchored primer may participate with one of the solution phase primers for amplification. Alternatively, a “generic” primer is anchored to the solid support in order to amplify the nucleic acid molecule comprising a target sequence. Specific amplification of the target sequence can then be achieved by solution phase primers. In relation to the latter embodiment, the solution would contain at least three solution phase primers wherein at least two primers would exhibit substantially complementarity with each other but differ by at least one mis-match.

The method of the present invention provides an efficient, cost effective and accurate means of detecting particular nucleic acid molecules. Such nucleic acid molecules may be derived from mammalian pathogens (e.g. hepatitis virus, human immune deficiency viruses, papilloma viruses and microorganisms such as Mycobacterium sp and Legionella sp or from plant pathogens (e.g. viruses, fungi or microorganisms).

Furthermore, the nucleic acid molecules may be derived from cancer tissue or from biopsy tissue from subjects with a particular disease condition or a suspected disease condition. Examples of such disease conditions associated with genetic mutation include but are not limited to sickle cell anaemia, phenylketonuria, Lesch-Nyhan syndrome, cystic fibrosis, thalassaemia, muscular dystrophy, Huntington's disease, leukaemia and a range of other cancers. The method of the present invention also has application in gene sequencing, forensic analysis, tissue typing, taxonomy and foetal sex determination.

The method of the present invention may, in particular, be used to detect nucleotide repeat number polymorphism such as occur with microsatellite polymorphisms. Such polymorphisms have been linked with neurodegenerative diseases such as Huntington's disease.

The method of the present invention has the capacity to reduce background amplification which can result in false positive or false negative results. In other words, the likelihood of mispriming is reduced.

In another embodiment, the competitive priming step can be undertaken after amplification with a generic primer. In this embodiment, a non-allele specific amplification is undertaken using an unlabelled primer. The amplimer is then interrogated using competitive priming on a solid phase. The use of a large amount of amplimer means that other minimal amplification is necessary.

According to this embodiment, there is provided a method for detecting a nucleic acid molecule having a particular nucleotide sequence anchored to a solid support via hybridization of said nucleic acid molecule to a primer anchored to said solid support, said method comprising subjecting said nucleic acid molecule to amplification using at least two solution phase primers and anchoring said amplified nucleic acid molecule to the solid support, contacting said anchored nucleic acid molecule with at least two other solution phase nucleic acid primers wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence with greater efficiency or specificity compared to the nucleic acid primer which contains a mis-match and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

More particularly, the present invention contemplates a method for detecting a nucleic acid molecule having a particular nucleotide sequence anchored to a solid support via hybridization of said nucleic acid molecule to a primer anchored to said solid support, said method comprising subjecting said nucleic acid molecule to amplification using at least two solution phase primers and anchoring said amplified nucleic acid molecule to the solid support, contacting said anchored nucleic acid molecule with at least two other solution phase nucleic acid primers wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence and to extend from its ₃′ terminus to form an extension product complementary to the stand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

Generally, the initial amplification using the first at least two primers is undertaken using non-labelled primers.

In another embodiment, the present invention contemplates conducting post-amplification interrogation using non-allele-specific primers with a high Tₘ and allele-specific primers with a low Tₘ. The initial amplification cycles are conducted under high annealing temperatures rendering the allelic specific primers inactive. The later cycles are conducted at a lower temperature thus activating the allele-specific primers. This embodiment has the convenience of the primers all being added at the beginning of the reaction. For particular convenience, the different annealing temperatures are programmed into the thermocycler. The Tₘ of the first set of primers (Tₘ₍₁₎) is greater than the Tₘ of the second set of primers (Tₘ₍₂₎) such that at the temperature employed for the amplification of the first set of primers, the second set of primers are inactive. Accordingly, the difference between Tₘ₍₁₎ and Tₘ₍₂₎ may be from about 3° C. to about 50° C. and more preferably from about 5° C. to about 20° C.

Accordingly, another aspect of the present invention contemplates a method for detecting a nucleic acid molecule having a particular nucleotide sequence anchored to a solid support via hybridization of said nucleic acid molecule to a primer anchored to said solid support, said method comprising subjecting said nucleic acid molecule to amplification using at least two solution phase primers having a high Tₘ and wherein the amplification conditions are such that said solution phase primers are active and anchoring said amplified nucleic acid molecule to the solid support, contacting said anchored nucleic acid molecule with at least two other solution phase nucleic acid primers wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said latter two primers have a Tₘ lower than the temperature used in said first amplification such that during the first amplification, the second set of primers is inactive but during said second amplification, said second set of primers is active, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

Preferably, there is a single immobilized primer of either low high Tₘ.

In an alternative embodiment, use is made of the fact that a low Tₘ primer is inactive at high annealing temperatures, but a high Tₘ primer still works at low temperatures. Furthermore, the immobilization of the amplimer due to immobilized primer extension can be during the first (non-allele specific) phase of the reaction as well as or in addition to the second allele specific phase. Therefore, the present invention extends to a single immobilized primer of either high or low Tₘ, a single solution phase primer of high Tₘ and two or more solution phase, allele specific primers of low Tₘ.

Accordingly, yet another aspect present invention contemplates a method for detecting a nucleic acid molecule having a particular nucleotide sequence anchored to a solid support via hybridization of said nucleic acid molecule to a primer anchored to said solid support, said method comprising subjecting said nucleic acid molecule to amplification using a solution phase primer having a high Tₘ and wherein the amplification conditions are such that said solution phase primers are active and anchoring said amplified nucleic acid molecule to the solid support, contacting said anchored nucleic acid molecule with at least two other solution phase nucleic acid primers each having a low Tₘ wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said latter two primers have a Tₘ lower than the temperature used in said first amplification such that during the first amplification, the second set of primers is inactive but during said second amplification, said second set of primers is active, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

The methods of the present invention may be used with respect to any form of amplification including polymerase chain reaction, ligation chain reaction, nucleic acid sequence based amplification, Qβ replicase based amplification, strand displacement method, rolling circle amplification and recirculating allele-specific primer extension.

In a further embodiment, a thermostable ligase may be employed with the non-allele specific amplification reaction. This results in the multimerization of the amplimer and this may improve the interrogation step. Preferably, a thermostable polymerase is also employed which does not put A-tails onto the amplimer. Alternatively, a T-tailed linker is used in the reaction. Either approach ensures that the amplimer monomers are ligatable. Although not wishing to limit the present invention to any one theory or mode of action, it is proposed that in certain circumstances, multimeric amplimers are more efficaciously interrogated compared to monomeric amplimers. The present invention extends to these modifications to the methodology but is not limited to these embodiments.

A range of labels providing a detectable signal may be employed. The label may be associated with a particular nucleic acid molecule or nucleotide or it may be attached to an intermediate which subsequently binds to a nucleic acid molecule or nucleotide.

The label may be selected from a group including a chromogen, a catalyst, an enzyme, a fluorophore, a luminescent molecule, a chemiluminescent molecule, a lanthanide ion such as Europium (Eu³⁴), a radioisotope and a direct visual label. In the case of a direct visual label, use may be made of a colloidal metallic or non-metallic particular, a dye particle, an enzyme or a substrate, an organic polymer, a latex particle, a liposome, or other vesicle containing a signal producing substance and the like. A large number of enzymes suitable for use as labels is disclosed in U.S. Pat. Nos. 4,366,241, 4,843,000 and 4,849,338. Suitable enzyme labels useful in the present invention include alkaline phosphatase, horseradish peroxidase, luciferase, β-galactosidase, glucose oxidase, lysozyme, malate dehydrogenase and the like. The enzyme label may be used alone or in combination with a second enzyme which is in solution. Alternatively, a fluorophore which may be used as a suitable label in accordance with the present invention includes, but is not limited to, fluorescein, rhodamine, Texas red, Lucifer yellow or R-phycoerytrin

Another aspect of the present invention extends to the use of arrays of nucleic acid primers immobilized to a solid support such as a microchip, microtiter well, dipstick, microscope slide or other suitable surface. The primers may be the same or may differ by one or more nucleotides. The immobilized primers may be used to amplify different regions of a nucleic acid molecule comprising a target sequence or may be directed to a range of different target sequences. The latter is useful in diagnosis of cancer, genetic diseases or for pathogen identification. The former is more useful in sequencing of a nucleic acid molecule. Reference herein to “arrays” is not to imply any particular order or arrangement and the “arrays” may comprise an ordered arrangement and/or a random arrangement of primers.

The immobilized primers are non-allele specific whereas the solution phase primers are allele specific.

Accordingly, another aspect of the present invention provides a solid support comprising an array of immobilized primers wherein each of the primers may comprise the identical nucleotide sequence or one or more may differ from each other by at least one nucleotide and wherein the array is used to detect a nucleic acid molecule having a particular nucleotide sequence immobilized to a solid support via hybridization of said nucleic acid molecule to said one or more primers anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence with greater efficiency and/or specificity compared to the nucleic acid primer which contains a mis-match and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

More particularly, the present invention provides a solid support comprising an array of immobilized primers wherein each of the primers may comprise the identical nucleotide sequence or one or more may differ from each other by at least one nucleotide and wherein the array is used to detect a nucleic acid molecule having a particular nucleotide sequence immobilized to a solid support via hybridization of said nucleic acid molecule to said one or more primers anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence but at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

In accordance with these embodiments, generally and in a preferred embodiment, the immoblized primers are non-allele specific whereas the solution phase primers are allele specific.

The method of the present invention may also be automated or semi-automated. This facilitates high through-put screening of samples. This embodiment further contemplates the use of microchip including biochip technology to facilitate the rapid screening of target nucleic acid molecules.

In practice, the methods of the present invention are preferably undertaken in multiples such as duplicates. In a duplicate, for example, one of the allele-specific solution phase primers is labelled in one reaction vessel whereas the other primer is labelled in another reaction vessel. The patter of +/− results from the label then determining which primer has been incorporated.

The present invention is also particularly useful in screening nucleotide sequences such as those arising in genome projects such as the human genome project. The techniques of the present invention, especially but not necessarily when automated, can be used to screen rapidly large amounts of sequences for particular genes of interest, potential transcription start sites and/or 5′ and ₃′ terminus regions of potential genes.

Although the method of the present invention is preferably conducted using a competitive priming post-amplification system, the instant invention further extends to non-competitive priming post-amplification conditions. Such an embodiment is conductible due to the small number of amplification cycles that would be carried out during the allele-specific phase of the reaction.

As stated above, the present invention is useful for the diagnosis of a range of conditions and in particular disease conditions having a causation factor involving repeat expansion. One particular example is neurodegenerative conditions such as Huntington's disease.

The instant assay enables the identification and/or discrimination between microsatellite repeat number alleles when the number of repeats of a microsatellite contained within an amplified DNA fragment is above or below a pre-defined threshold value.

The basis of this method is that a primer that contains a large number of repeat units and an anchor sequence at the 5′ end can only hybridize to the target to yield an extendable 3′ end if a non-hybridized loop forms in the primer. It is expected that a large proportion of annealed primer would have non-hybridized 3′ end. Conversely, a primer that differs from the “many repeats” primer in that it contains a smaller number of repeating units than the target can anneal to this target and yield an extendable 3′ end without loop formation. If, however, the target possesses an equal or greater number of repeats than the “many repeats” primer, then the “many repeats” primer can also anneal and yield an extendable 3′ end without loop formation.

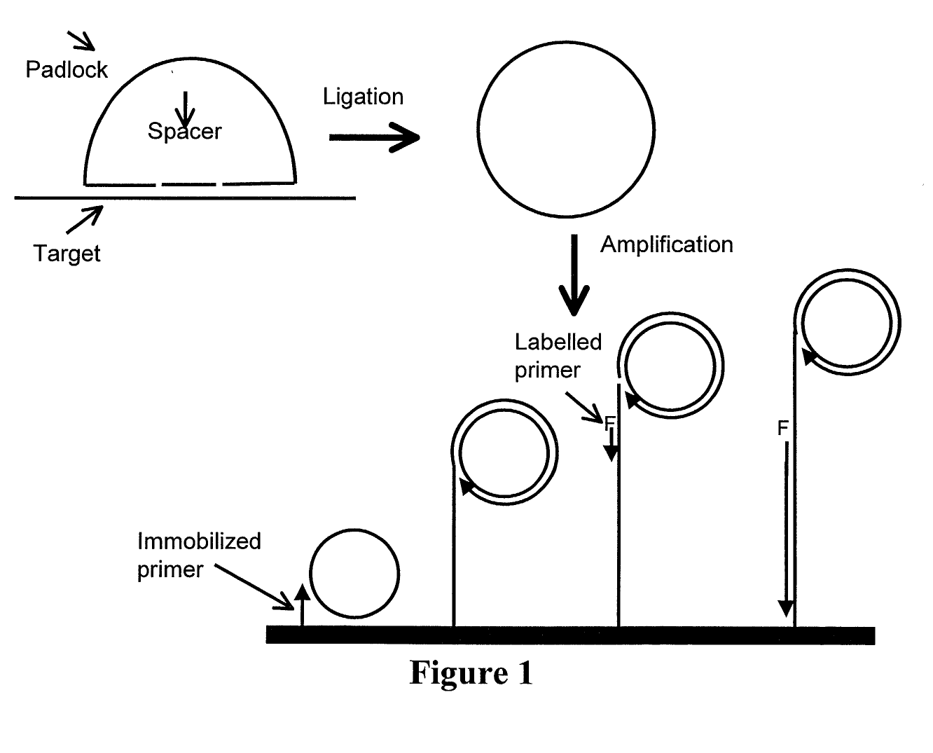

The ΔG of a hybridization reaction is proportional to the number of hydrogen bonds that form. Consequently, if the primers are present at the appropriate concentrations, the “many repeats” primer would out compete the “few repeats” primer when the number of repeats in the target is greater than the threshold and, conversely, the “few repeats primer” out-competes the “many repeats” primer when the number of repeats in the target is fewer than the threshold. The reaction is depicted in FIG. 1.

Because long primers have an inherently greater affinity for the target than short primers, it may be difficult to achieve a differential reaction when the target has a low number of repeats. However, when the target has a high number of repeats, the “many repeats” primer will have a strong competitive advantage. In this situation, it is still possible to discriminate heterozygous and homozygous mutants from homozygous normals since the “many repeats” primer will have a competitive advantage whenever a “many repeats” allele is in the sample.

This strategy is adaptable to any DNA diagnostic format that relies upon competitive primer extension. The primers may also be used as PCR primers so that amplification and discrimination are carried out in a single step. Alternatively, amplified material can be interrogated using this method. Because of the ubiquity of microsatellite loci with similar repeating units in the genomes of higher organisms, in some situations, it might be difficult to achieve amplification specificity in a single step procedure. Therefore, in a preferred embodiment, the assay involves an interrogation of amplified microsatellite loci. This interrogation may be done by:

1. Solid phase amplification (SPA): In this embodiment, the discriminatory primers are in the solution phase and a “common” primer is immobilized to a solid phase. A limited amplification reaction is then carried out the number of repeats specifies which solution phase primer is incorporated into the amplimer. The amplimer is localized to the solid phase by the extension of the nondiscriminatory solution phase primer.

2. Solid phase nucleic acid amplification and capture (SNAAC): This embodiment differs from SPA in that all the amplification primers are in the solution phase and the amplimer is captured by an immobilized oligonucleotide that is designed to be non-extendable and to be complimentary to a part of the amplimer not encompassed by the primer sequences. In some circumstances this may increase the specificity because any spurious amplimer synthesised will not be captured.

3. Differential capture: In this embodiment, there are no immobilized oligonucleotides and the primers are differentially labelled with capture probes rather than detection probes. The primary amplimer can be interrogated by either a limited exponential amplification or by a single extension reaction. Any extension products derived from the labelled primer are captured and detected, while extension products derived from the unlabelled primer are washed away. Detection can be by means of a labelled dNTP that is incorporated in the amplimer or by means of a double-stranded DNA specific fluorescent dye.

The solid-phase/competitive priming reactions described herein involve allele specific amplimer labelling and non-allele specific amplimer capture.

In an alternative embodiment, the instant method makes use of allele specific amplimer labelling with a capture label followed by capture then non-allele specific detection of captured amplimer.

One example of a capture label is biotin which can be captured onto streptavidin coated micro-wells or magnetic beads.

Amplimer detection can be by detection of labelled dNTP which is incorporated in the amplification reaction. An example of a label that could be used is digoxigenin. Alternatively, the amplimer could be detected using a double-strand specific fluorescent dye such as Cybr green.

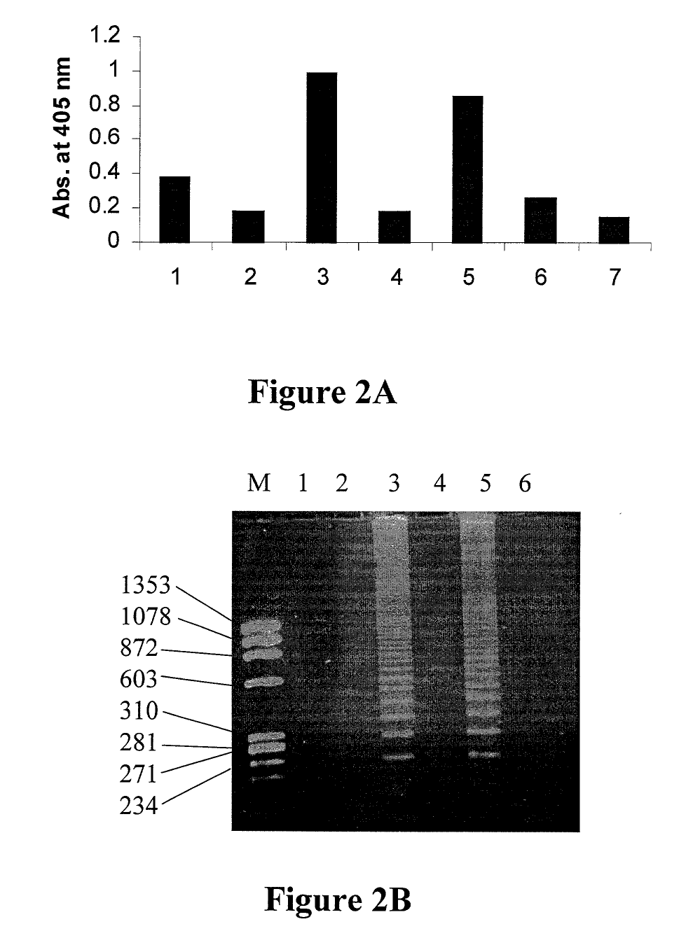

The procedure is depicted in FIG. 2.

The present invention extends to a non-competitively primed, solid phase embodiment. In this embodiment, the reactions comprise a solid phase primer and a single solution phase allele specific primer.

Furthermore, the present invention encompasses the situation where the amplimer is captured by a non-extendable immobilized oligonucleotide instead of by one of the amplification primers.

The present invention farther contemplates target quantification. According to this embodiment, a target nucleic acid molecule is amplified using competitive primers wherein at least one is a perfect match relative to the target and at least one other contains a single mis-match. The amount of target available determines the number of cycles before amplification stops. Consequently, the lower the concentration of target, the more amplification cycles before amplification ceases and, hence, the less proportional difference there is between the incorporation of the matched primer and the mis-matched primer. Given a suitable standard curve, the amount of target is calculated from the ratio of the incorporation of the matched and mis-matched primers at the end of the amplification reaction.

Accordingly, another aspect of the present invention contemplates a method for quantitating a nucleic acid molecule immobilized to a solid support via hybridization of said nucleic acid molecule to a primer anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primers to amplify said target sequence to completion of an amplification reaction wherein the amount of target nucleic acid molecule is proportional to the number of amplification cycles such that the amount of target nucleic acid molecule is determined from the ratio of incorporation of complementary and mis-match primers at the end of the amplification reaction.

Preferably, two solution phase primers are used, being a perfect match primer and a single nucleotide mis-matched primer.

The use of competitive priming to quantitate target nucleic acid molecules is predicated in part on the relationship between the number of amplification cycles and the proportion of the amplimer which is mis-primed product. When amplification is exponential and 100% efficient, i.e. the amount of amplimer doubles with each cycle or unit of time, the relationship may be represented as follows:

$$\frac{b}{t o t a l} = \frac{n - 1}{4 M {e}^{\frac{( n - 2 )}{4 M}}}$$

wherein:

b=homoduplex DNA that is the opposite allele as the target (i.e. amount of mis-primed product)

total=amount of amplimer

n=PCR cycle number

M=mis-priming frequency written as a whole number

e=the exponential constant (2.718281828).

This function may be graphically plotted and shows that in a competitive priming reaction, the more cycles of amplification, the greater the proportion of mis-primed product in comparison to total amplimer.

When there is an unknown amount of target, the number of amplification cycles will be a function of the amount of target, the more target the fewer cycles before the reaction runs out of monomers as the maximum amount of amplimer that can be synthesized is reached. Therefore, the (b/total) figure is a function of the amount of target. Consequently, b/total is used to calculate the amount of target.

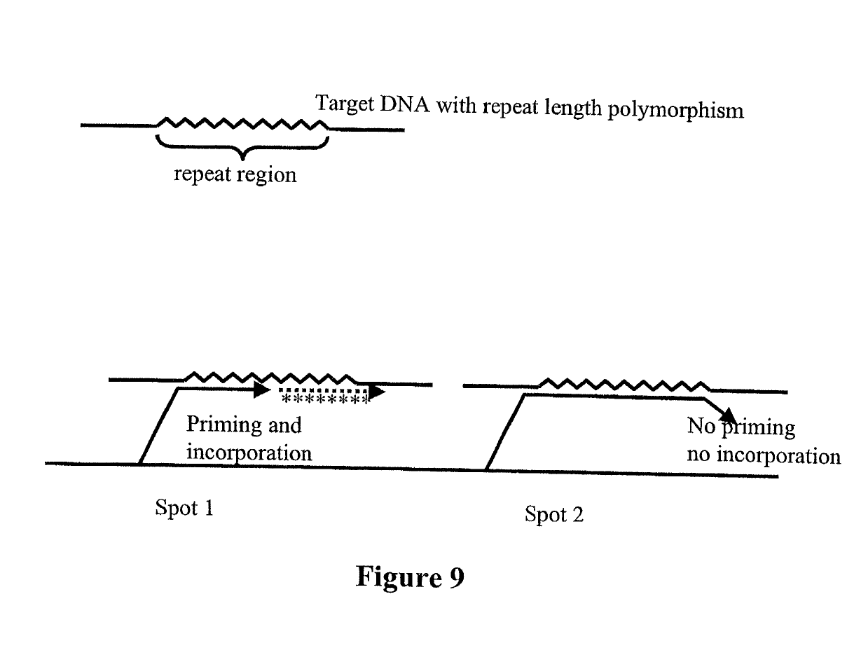

In another embodiment of the present invention, a pair of solid phase oligonucleotides are used to measure nucleotide repeat number polymorphism including microsatellite repeat sequences. The primers may be in separate wells or on separate array spots. The target nucleic acid molecule is then subjected to non-competitive primer extension. The extension is at or near the nucleotide length polymorphism or microsatellite. Consequently, one primer is capable of inducing extension whereas the other primer is not. By using incorporation of a label, the extension or lack of extension is thereby detectable. This concept is shown diagrammatically in FIG. 9. In this depiction, a target DNA molecule comprises a repeat region indicated by a wavy line. The target DNA is subjected to interrogation using a pair of primers where one primer is capable of priming and label incorporation and the other primer is not, depending on the presence or, absence of the repeat regions.

Consequently, another aspect of the present invention contemplates a method for detecting a nucleotide length polymorphism in a target nucleic acid molecule, said method comprising subjecting said target nucleic acid molecule to interrogation by a pair of immobilized primers wherein said primers are immobilized in separate reaction vessels or separate spot arrays and wherein one primer is capable of priming a particular nucleotide length polymorphism whereas the other primer is unable to induce priming of said nucleotide length polymorphism wherein at least one primer is labelled with a reporter molecule capable of providing an identifiable signal wherein the relative presence or absence of said signal is indicative of the presence or absence of said nucleotide length polymorphism.

In one embodiment, the nucleotide length polymorphism is microsatellite DNA.

In a particularly preferred embodiment, the nucleotide length polymorphism or microsatellite DNA is indicative of the presence or absence of a particular disease condition or a propensity for development of said disease condition such as but not limited to a neurodegenerative disorder (i.e. Huntington's disease).

The present invention is further described by the following non-limiting Examples.

### EXAMPLE 1

### Interrogation of Multimeric DNA Amplification Products by Competitive Primer Extension

Linear double stranded DNA composed of tandem repeats may be exponentially amplified by the strongly strand displacing Bst DNA polymerase (large fragment) and two primers specific for opposite strands. When the repetitive DNA is derived from rolling circle replication of a circular template, the reaction is termed cascade rolling circle amplification (CRCA). The inventors developed a variant of CRCA in which one primer is attached to the surface of a micro-well and the other is labelled, thus enabling detection of amplified material using an ELISA-like protocol. The circular template is derived by annealing and ligation of a padlock on target DNA. There is good correlation between the synthesis of amplified material and signal.

Oligonucleotide Synthesis

Oligonucleotides were supplied by Gibco BRL (now Invitrogen, Mulgrave, Victoria, Australia) or by Bresatec (now GeneWorks, Adelaide, South Australia, Australia). Oligonucleotides used as padlocks and targets were gel purified to homogeneity by the suppliers. All other oligonucleotides were supplied as desalted preparations.

Oligonucleotide Sequences Derived from Chlamydia

Padlocks were designed essentially as described by Nillson et al. (10). They were based upon the reported sequences for the groEL genes from Chlamydia trachomatis and Chlamydia pneumoniae (11, 12).

The Chlamydia-specific padlock was: 5′GCAGGTAAAGAAGGCGCCGCGGTGAGCTATATGGGGACTATGAATTTGCTCCATTAAAGCAAATTGC3′ [SEQ ID NO:12]. The bases designed to hybridize to either or both of the targets are single underlined, the bases designed to hybridize to the immobilized common primer FVComT are double underlined, and the region identical to the allele specific primers (minus 4 bases at the 3′ ends which are variable and terminate in the spacers) is in bold.

The Chlamydia pneumoniae-derived target was: 5′ATAGCGCCTTCTTTACCTGCGTTACTTCGAATTTGCTTTAATGGAGCTG3′ [SEQ ID NO:13]. The bases designed to hybridize to the padlock are underlined.

The C. pneumoniae-specific spacer was: 5′AAGTAAC3′ [SEQ ID NO:14]. The T was biotinylated, but this was not used as a capture tag in any experiments.

The Chlamydia trachomatis-derived target was: 5′ATAGCACCTTCTTTTCCTGCGTTTGCACGAATTTGTTTCAAAGGAGCGG3′ [SEQ ID NO:15]. The bases designed to hybridize to the padlock are underlined. The C. trachomatis-specific spacer was:5′TGCAAAC3′ [SEQ ID NO:16]. The T was biotinylated, but this was not used as a capture tag in any experiments.

The C. pneumoniae and C. trachomatis-specific amplification primers were each designed to terminate within the corresponding spacer sequences. They were of sequence 5′CCATTAAAGCAAATTGCAAG3′ [SEQ ID NO:17] and 5′CCATTAAAGCAAATTGCTGCA3′ [SEQ ID NO:18] respectively and labelled with 6-carboxy fluorescein (FAM) at the 5′ C residues. The non-allele specific primer (FVComT) was immobilized to the micro-well wall. It was designed to be complementary to the padlock and to incorporate a poly-T spacer sequence at the 5′ end. Its sequence was 5′TTTTTTTTTGTCCCCATATAGCTCACCG3′ [SEQ ID NO:5].

Oligonucleotide Sequences Derived from the Factor V Leiden Mutation Site (13)

The padlock was: 5′ AGGAATACAGGTATTTTGTCCTTGCGCTGGGGACTATGAATTTCTAATAGGACTACTTCTAATCTGTAAGAG3′ [SEQ ID NO:3]. The regions that hybridize to the target are single underlined and the region complementary to the common primer FVComT is double underlined.

The spacer specific for the wild-type sequence was 5′CAGATCCCTGGACAGGCG3′ [SEQ ID NO:19].

The spacer specific for the mutant sequence was 5′CAGATCCCTGGACAGGCA3′ [SEQ ID NO:20].

The FAM labelled allele specific primers were of the same sequences as the spacers except that sub-terminal mis-matches are incorporated. They were 5′CAGATCCCTGGACAGACG3′ [SEQ ID NO:6] (wt) and 5′CAGATCCCTGGACAGACA3′ [SEQ ID NO:7] (mutant) and with labels at the 5′ terminal C residues. The unlabelled primers were the same sequence but without the 5′ terminal CA residues. The non-allele specific immobilized primer used was FVComT as for the Chlamydia derived system.

Binding of FVComT to Microwells

NUNC NucleoLink Strips (Cat No: 248259, obtained from Medos Company Pty. Ltd., Mt. Waverley, Victoria, Australia) were used throughout. To each well was added 100 μl of 100 nM FVComT, 10 mM 1-ethyl-3-(3-dimethylaminopropyl)-carbodiimide, 10 mM 1-methyl-imidiazole. The wells were sealed with cling-film and incubated at 50° C. overnight. The solution was then removed and the wells washed three times with 100 mM Tris-HCl pH 7.5, 150 mM NaCl, 0.1% v/v Tween 20 (TBST), soaked for five min then washed three more times with the same buffer. Finally, the wells were washed once with deionized water and stored at 4° C.

Padlock Circularization

Target DNA was mixed with 1 pmole padlock, 1 pmole spacer molecule and 5U Ampligase DNA ligase in Ampligase buffer (both from Epicentre Technologies, Austral Scientific, Gymea, NSW Australia) in a total volume of 50 μl. The mixture was heated to 94° C. for 3 min then incubated at 60° C. for 20 min.

Primary and Single Step Amplifcation Reactions

Each reaction contained 1 μl of ligation reaction products, 10 mM dNTPs (Boehringer Manmheim), 166 nM solution phase primers made up to a final volume of 60 μl in thermopolymerase buffer (New England Biolabs, Genesearch Pty. Ltd. Arundel, Queensland, Australia). The mixture was heated to 94° C. for 3 mnin then 4 U Bst DNA polymerase (large fragment) (New England Biolabs, Genesearch Pty. Ltd. Arundel, Queensland, Australia) added and the reaction incubated for 90-120 min at 60° C.

Secondary Amplification Reactions

These were carried out in Nunc micro-wells coated with primer FVComT as described above. The reaction conditions were as for the primary amplification reactions except that the solution phase primer were added to 333 nM, the substrates were reaction products from primary amplification reactions and the incubation was for 10 min at 60° C.

When the primary amplification was carried out in a microfuge tube, 10 μl of the primary amplification products were added to the secondary amplification reaction. When the primary amplification was carried out in a micro-well, the allele specific primers and a further 4 U Bst polymerase (large fragment) were added to the primary amplification products.

Quantitation of Immobilized FAM-labelled Primer

The solution phase amplification products were removed from the wells which were then washed three times with TBST at room temperature, twice with TBST at 80° C. (incorporating a 2 min incubation in the presence of each aliquot of TBST) then once more with TBST at room temperature. The final wash solution was removed and the wells blocked with 50 μl 10 mg/ml BSA for one hour then washed three times with TBST. 50 μl 1:3000 diluted anti-fluorescein antibody-alkaline phosphatase conjugate (Boehringer Mannheim) was added to the wells which were then incubated at room temperature for 30 minutes. The antibody solution was removed and the wells washed 6 times with TBST at room temperature then twice with 100 mM Tris-HCl pH 7.5, 150 mM NaCl at room temperature. 50 μl para-nitrophenylene phosphate (PNPP) (Sigma) solution (1 mg/ml in the buffer provided by the manufacturer) was then added to the wells which were incubated in the dark for 30 minutes. The absorbances were then read at 405 nm.

Adaption of Cascade Rolling Circle Amplification (CRCA) to the Solid Phase

The solid phase CRCA method is illustrated in FIG. 1.

Initial experiments to develop solid phase CRCA reactions made use of synthetic targets derived from the hsp60 genes of Chlamydia trachomatis and Chlamydia pneumoniae. A synthetic padlock was designed to be able to anneal to both targets leaving a 6 base gap between the padlock ends. Species specific spacer molecules were designed to be able to anneal within this gap leaving single strand breaks. Thus, ligase-mediated padlock circularisation would be expected to occur only when the spacer sequence was complementary with the target, and the circularized padlock molecule would be expected to incorporate the spacer.

It can be seen in FIG. 2 that detectable amplified material was synthesized only when the spacer and target were homologous. It can also be seen that the presence of detectable amplified material corresponded to high absorbance readings from the assays.

This reaction is termed herein solid phase cascade rolling circle amplification (SPCRCA).

Post Amplification Competitive Priming Provides Single Nucleotide Specificity

A competitive priming strategy was adopted which included two allele specific primers in the reaction. Amplified material is analyzed to determine which primer had been incorporated. The devisal experimental system made use of pairs of reactions in which one allele specific primer was labelled and the other not labelled.

It was recognized that in a competitively primed amplification reaction there is a direct relationship between the degree of amplification and proportion of total amplified material that is a product of primer mis-incorporation. This is because as amplification proceeds, any products of mis-priming are replicated while new mis-priming events occur. Therefore, it was predicted that reducing the extent of the allele specific amplification would increase its specificity.

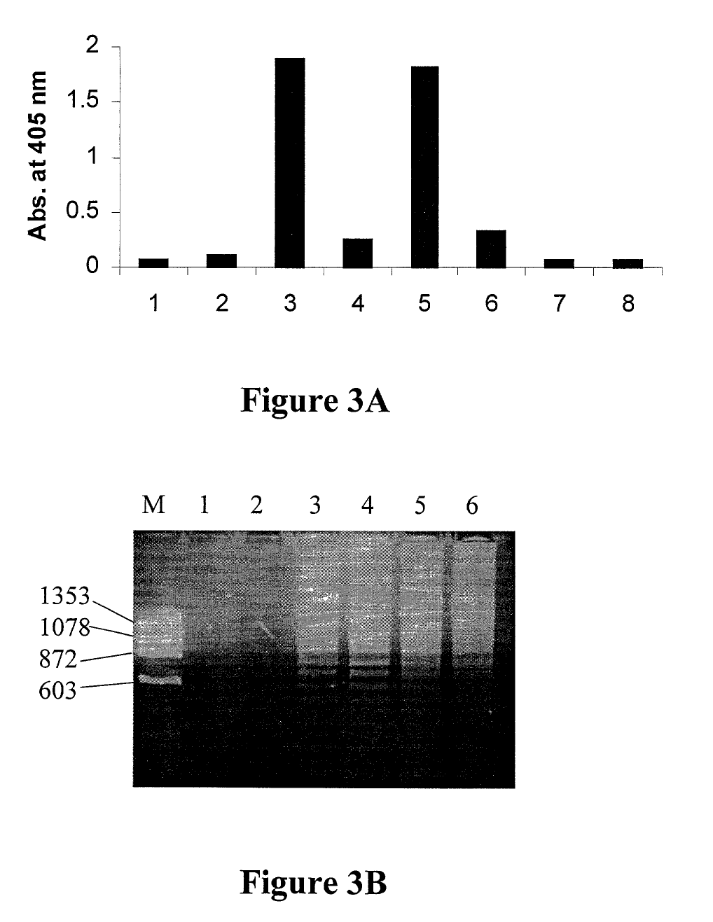

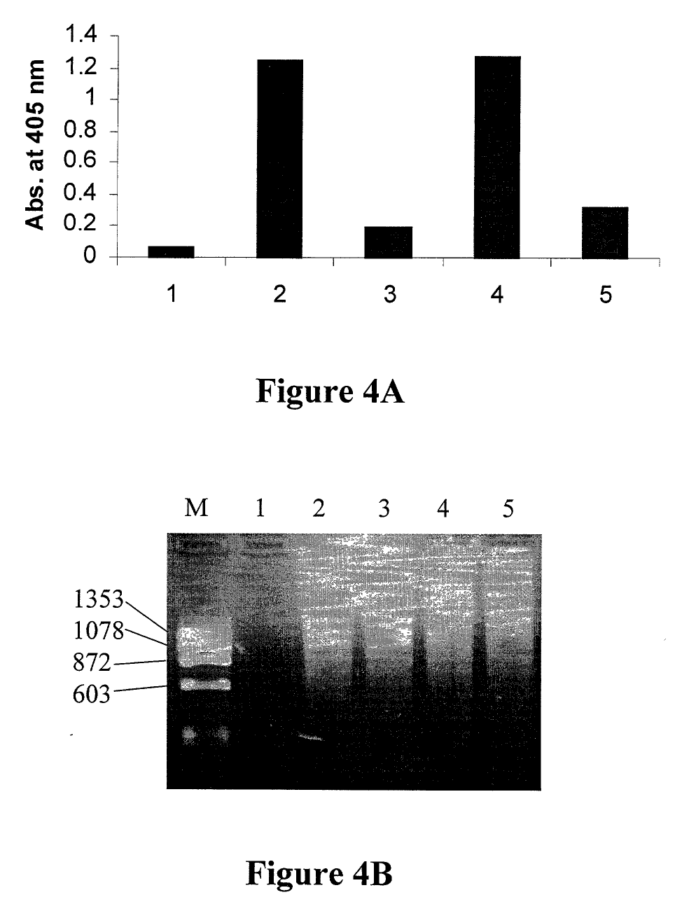

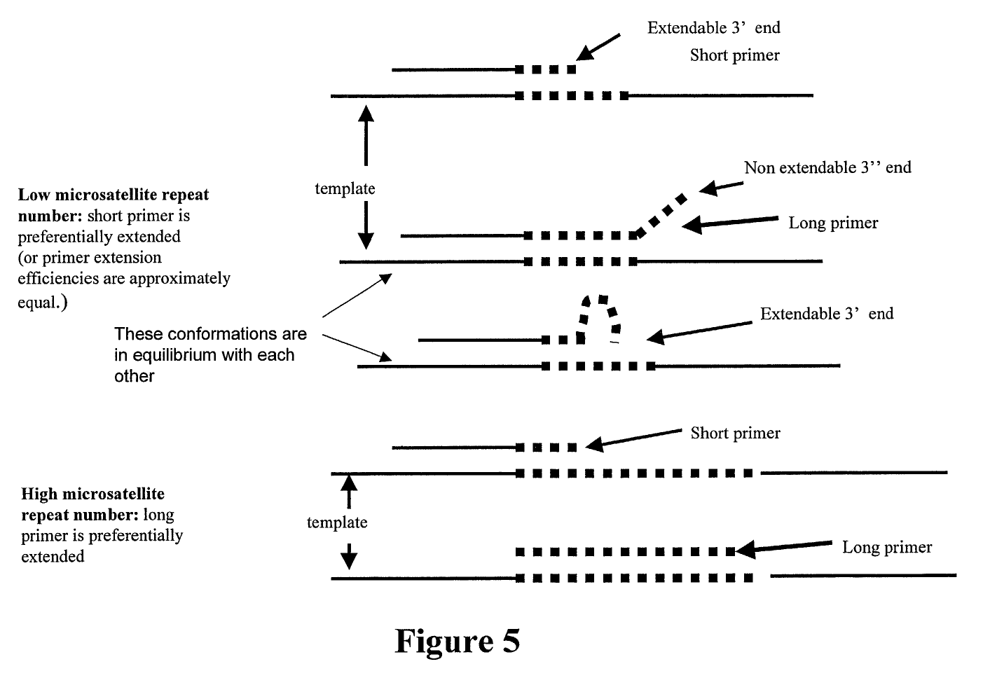

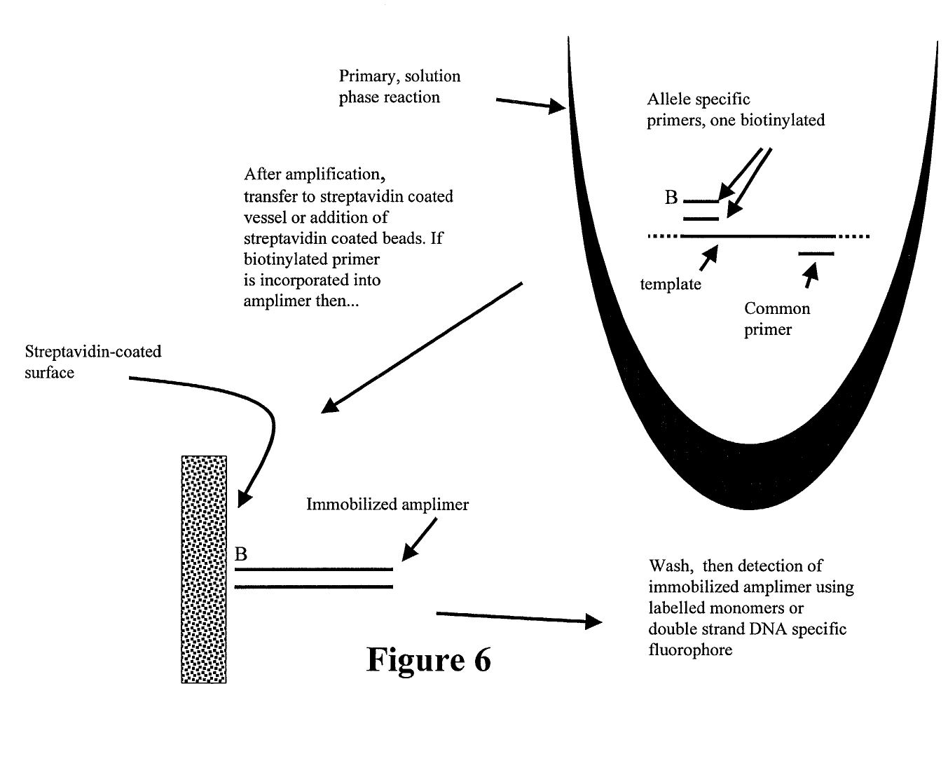

This was found to be the case. If the circular template was first amplified by CRCA in a non-allele specific manner and the amplified material then interrogated by competitive priming for 10 mnin under similar reaction conditions, the relative incorporation of the allele specific primers was in accordance with the allele present in the circular template. This experiment was initially performed such that the first amplification reaction was in a microfuge tube and the amplified material interrogation in a micro-well. High absorbance readings were obtained from the positive reactions, indicating that the 10 min amplified material detection step was sufficient for significant priming from the immobilised primer (FIG. 3). Similar results were obtained when both amplification reactions were carried out in the same micro-well and a genomic target was used (FIG. 4). The similar quantity of solution phase amplified material in the different reactions illustrates that the specificity observed was due to differential primer incorporation rather than differential degrees of amplification. Allele specificity was found to be robust upon repetition of the reactions, and careful optimisation of primer concentration was not necessary.

### EXAMPLE 2

### Comparison of the Specificity of Competitive Priminig and Non-competitive Priming in a Rolling Circle Solid Phase Amplification (RC-SPA) Reaction

The purpose of this experiment was to compare the specificity of competitive priming and non-competitive priming strategies in an RC-SPA reaction.

In this case, a post-amplification competitive priming strategy was not used. Instead single step RC-SPA reactions were carried out, i.e. the allele specific primers were present from the beginning of the amplification reaction.

A synthetic target derived from the human Factor V gene and encompassing the site of the Leiden mutation was used as the target. This was used to circularize a padlock probe-spacer mixture. A competitive ligation reaction was employed spacers corresponding to both the Leiden mutation and the wild-type sequences were included in the ligation reaction.

Procedure

Binding of the Non-allele Specific Primer FVComT2 to the Wells in Nunc Microtiter Plates

To each well was added 100 μl of 100 mM FVComT2 [SEQ ID NO:5], 10 mM 1-ethyl-3-(3-dimethylaminopropyl)-carbodiimide, 10 mM 1-methyl-imidazole. The wells were sealed with cling-film and incubated at 50° C. overnight. The coating solution was then removed and the wells washed three times, soaked for five min then washed three more times with 100 mM Tris-HCl pH 7.5, 150 mM NaCl, 0.1% v/v Tween 20 (TBST). Finally the wells were washed once with deionised water and stored at 4° C.

Ligation

1 fmole of wild type or mutant target molecules was mixed with 1 pmole padlock, 1 pmole wild type-specific spacer, 1 pmole mutant-specific spacer, and 5 U ampligase DNA ligase (Epicentre Technologies) in a total volume of 50 μl ampligase buffer (Epicentre Technologies). The mixture was heated to 94° C. for 3 min then incubated at 60° C. for 20 min.

Competitive RC-SPA

Nunc microtiter plate wells coated with primer FVComT2 [SEQ ID NO:5] were blocked with 50 μl 10 mg/ml bovine serum albumin (BSA) for one hour. The blocking solution was removed and 60 μl of a solution containing 10 mM dNTrs (Boehringer Mannheim), 20 pmoles FVComT2 [SEQ ID NO:5], 20 pmoles labelled FVW2 [SEQ ID NO:6] or FVM2 [SEQ ID NO:7], 20 pmoles unlabelled FV5 [SEQ ID NO:8] or FV6 [SEQ ID NO:9] and 1 μl ligation mixture in 1×thermopolymerase buffer (New England Biolabs) added to the well. This was then heated at 94° C. for 3 min then 4 U Bst DNA polymerase large fragment (New England Biolabs) was added and the reaction incubated at 60° C. All incubations of the microtiter plates were carried out using an MJ Research Thermal Cycler-200.

Quantitation of Immobilized Labelled Primer

The solution phase amplification products were removed from the wells which were then washed three times with TBST at room temperature, twice with TBST at 80° C. (incorporating a 2 min incubation in the presence of each aliquot of TBST) then once more with TBST at room temperature. The final wash solution was removed and the wells blocked with 50 μl 10 mglml BSA for one hour then washed three times with TBST. 50 μl 1:3000 diluted anti-fluorescein antibody-alkaline phosphatase conjugate (Boehringer Mannheim) was then added to the wells which were then incubated at room temperature for 30 minutes. The antibody solution was removed and the wells washed 6 times with TBST at room temperature then twice with 100 mM Tris-HCl pH 7.5, 150 mM NaCl at room temperature. 50 μl para-nitrophenylene phosphate (PNPP) (Sigma) solution (1 mg/ml in the buffer provided by the manufacturer) was then added to the wells which were then incubated in the dark for 30 minutes. The absorbances were then read at 405 nm.

Results

The A₄₀₅ readings are shown in Table 1.

TABLE 1

| | -tem | -lig | w+ | w− | m+ | m− |
|---|---|---|---|---|---|---|
| comp | 0.339 | 0.350 | 2.366 | 0.281 | 1.704 | 2.203 |
| non-comp | 2.183 | 2.466 | 2.798 | 2.581 | 2.444 | 2.533 |

−tem No template, labelled mutant specific primer (FVM2 [SEQ ID NO:7]) and unlabelled wild-type specific primer (FV5 [SEQ ID NO:8])

−lig Mutant template, no ampligase, primers as for −tem

w+ Wild-type target, labelled wild-type specific primer (FVW2 [SEQ ID NO:6]), (unlabelled mutant-specific primer (FV6) [SEQ ID NO:9])

we− Wild-type target, labelled mutant specific primer (FVM2 [SEQ ID NO:7]), (unlabelled wild-type specific primer (FV5) [SEQ ID NO:8])

m+ Mutant target, labelled mutant specific primer VM2 [SEQ ID NO:7]), (unlabelled wild-type specific primer (FV5) [SEQ ID NO:8)

m− Mutant target, labelled wild-type specific primer (FVW2 [SEQ ID NO:6]), (unlabelled mutant specific primer (FV6) [SEQ ID NO:9])

comp Competitive priming used—the unlabelled primers in brackets were included in the reaction

non-comp A non-competitive priming strategy was used—the unlabelled primers in brackets were not included in the reaction

It can be seen that in the case of the wild-type target, the competitive priming strategy provided allele specificity whereas the non-competitive strategy did not. In the case of the mutant target, however, the readings from the two alleles were approximately equal. This demonstrates that the specificity evident when the wild-type target was used was not simply due to a higher priming efficiency of the wild-type specific primer. It also demonstrates the difficulty of achieving allele specificity when a single-strep procedure is employed i.e. when there is only one amplification reaction and this contains the allele specific primers.

### EXAMPLE 3

In this example, the discriminatory power of post amplification RC-SPA in combination with competitive priming is demonstrated. In order to ensure that competitive ligation reactions had no confounding effects on the results, all ligations contained only one spacer molecule and that was perfectly complimentary to the target sequence used. A genomic template rather than a synthetic target was used and the mutant genomic sample was heterozygous but the spacer complementary to the mutant allele was used at all times with the mutant samples.

Procedure

Ligations

300-500 ng of wild type or mutant target molecules was mixed with 1 pmole padlock, 1 pmole wild type-specific or mutant specific and 5 U ampligase DNA ligase (Epicentre Technologies) in 1×ampligase buffer (Epicentre Technologies) in a total volume of 50 μl. The mixture was heated to 94° C. for 3 min then incubated at 60° C. for 20 min. The reactions were carried out in Eppendorf tubes.

First (Non-allele Specific) Amplification

This was carried out in Eppendorf tubes. The reactions contained 10 mM dNTP's (Boehringer Mannheim), 10 pmoles primer FVCom2, 10 pmoles primer FVA [SEQ ID NO:4], and 1 μl of the product from the ligation reaction in a final volume of 60 μl thermopolymerase buffer (New England Biolabs). The mixture was heated to 94° C. for three minutes then 4 U Bst polymerase large fragment added and the reaction incubated at 60° C. for 1 h 40 min.

Allele Specific RC-SPA

This reaction was carried out in Nunc wells that had previously been coated with primer FvComT as outlined in Example 2. The amplification reaction contained 10 mM dNTP's (Boehringer Manmheim), 2 pmoles primer FVComT2 [SEQ ID NO:5], 20 pmoles labelled FVW2 or FVM2 [SEQ ID NO:7], 20 pmoles FV5 or FV6 and 10 μl of the reaction products from the non-allele specific amplification in a final volume of 60 μl thermopolymerase buffer (ew England Biolabs). The mixture was heated to 94° C. for three minutes then 4 U Bst polymerase large fragment added and the reaction incubated at 60° C. for 10 min.

Quantitation of Immobilized Labelled Primer

This was carried out as described in Examples 1 and 2.

Results

The A₄₀₅ readings are in Table 2.

TABLE 2

| | -ve | -ve | w+ | w− | m+ | m− |
|---|---|---|---|---|---|---|
| 10 min | 0.050 | 0.323 | 1.047 | 0.155 | 0.955 | 0.184 |
| 30 min | 0.076 | 0.699 | 2.234 | 0.306 | 2.229 | 0.429 |

−tem1 Second amplification reaction only, carried out in the absence of target DNA. template. Labelled mutant specific primer and unlabelled wild-type specific primer.

−tem2 Both amplification reactions carried out, with material from first reaction added to second allele specific reaction. No target DNA present. Labelled mutant specific primer and unlabelled wild-type specific primer.

w+ Wild-type target, labelled wild-type specific primer, unlabelled mutant specific primer

w− Wild-type target, labelled mutant specific primer, unlabelled wild-type specific primer

m+ Mutant target, labelled mutant specific primer, unlabelled wild-type specific primer

m− Mutant target, labelled wild-type specific primer, unlabelled mutant specific primer

10 min 10 minutes colour development

30 min 30 minutes colour development

It is evident that the allele specific competitively primed RC-SPA reactions have single-nucleotide specificity with templates consisting of amplimers containing either wild-type or mutant sequences.

### EXAMPLE 4

This demonstrates that both the initial non-allele specific and final allele specific amplification reactions can be carried out in a Nunc well. In this case, the amplimer is immobilized to the side of the well during both the primary and secondary amplification reactions i.e. both the primary and secondary amplifications are RC-SPA. As for Example 1 and 3, genomic target samples and single spacer molecules were used.

Procedure

Ligation

As for Example 3.

First (Non-allele Specific) RC-SPA

This reaction was carried out in Nunc wells that had previously been coated with primer FvComT as outlined in Examples 1 and 2 then blocked with 50 μl 10 mg/ml BSA for 1 h. The reactants in the solution phase were as described in Example 3.

Allele Specific RC-SPA

20 pmoles of labelled FVW2 [SEQ ID NO:6] or FVM2 [SEQ ID NO:7] and 20 pmoles unlabelled FV5 [SEQ ID NO:8] or FV6 [SEQ ID NO:9] were added to material in the wells. The wells were then heated at 94° C. for three minutes, then 4 U Bst polymerase large fragment (ew England Biolabs) and the wells incubated at 60° C. for 10 min.

Quantitation of Immobilized Labelled Primer

This was as described in Examples 1 and 2.

Results

The A₄₀₅ readings are in Table 3.

TABLE 3

| | -tem | -lig | w+ | w− | m+ | m− |
|---|---|---|---|---|---|---|
| 10 min | 0.064 | 0.064 | 1.057 | 0.134 | 0.675 | 0.106 |
| 30 min | 0.068 | 0.066 | 2.583 | 0.247 | 1.687 | 0.173 |

−tem No template, labelled mutant specific primer and unlabelled wild-type specific primer

−lig 500 ng genomic mutant template, no ligase, labelled mutant specific primer, unlabelled wild-type specific primer

w+ 300 ng genomic wild-type target, labelled wild-type specific primer, unlabelled mutant specific primer

w− 300 ng genomic wild-type target, labelled mutant specific primer, unlabelled wild-type specific primer

m+ 500 ng genomic mutant target, labelled mutant specific primer, unlabelled wild-type specific primer

nm− 500 ng genomic mutant target, labelled wild-type specific primer, unlabelled mutant specific primer

10 min 10 min colour development

30 min 30 min colour development

It is evident that this procedure gave very similar results to those from Example 3, in which the primary amplification reactions were carried out in Eppendorf tubes. Therefore, carrying out the primary amplification reaction in Nunc wells does not compromise the secondary allele specific RC-SPA reactions.

### EXAMPLE 5

An identical procedure to that described in Example 4 was carried out, with the exception that a non-competitive priming strategy was used for the secondary allele-specific RC-SPA reaction.

Results

The A₄₀₅ readings are in Table 4

TABLE 4

| | -tem | -lig | w+ | w− | m+ | m− |
|---|---|---|---|---|---|---|
| 10 min | 0.052 | 0.050 | 0.576 | 0.110 | 0.597 | 0.335 |
| 30 min | 0.069 | 0.063 | 1.511 | 0.232 | 1.447 | 0.870 |

−tem No template labelled mutant specific primer

−lig 500 ng genomic mutant template, no ampligase, labelled mutant specific primer

w+ 300 ng genomic wild-type target, labelled wild-type specific primer

w− 300 ng genomic wild-type target, labelled mutant specific primer

m+ 500 ng genomic mutant target, labelled mutant specific primer

m− 500 ng genomic mutant target, labelled wild-type specific primer

It is clear that the specificity of the RC-SPA reactions using the mutant template is much poorer (and the specificity using the wild-type template slightly poorer) than the specificity obtained in Example 4, thus indicating the improvement in specificity provided by making use of competitive priming.

### EXAMPLE 6

An identical procedure to that described in Example 4 was carried out, with the exception that two different concentrations of allele specific primers were used.

Results

The A₄₀₅ readings are shown in Table 5.

TABLE 5

| | -tem | -lig | w+ | w− | m+ | m− | |
|---|---|---|---|---|---|---|---|
| 10 min | 0.081 | 0.090 | 0.449 | 0.105 | 0.684 | 0.220 | 10 pmoles |
| | | | | | | | primer |
| 10 min | | | 0.650 | 0.110 | 0.601 | 0.211 | 30 pmoles |
| | | | | | | | primers |

−tem No template, labelled mutant specific primer and unlabelled wild-type specific primer

−lig Genomic mutant template, no ampligase, labelled mutant specific primer, unlabelled wild-type specific primer

w+ Genomic wild-type target, labelled wild-type specific primer, unlabelled mutant specific primer

w− Genomic wild-type target, labelled mutant specific primer, unlabelled wild-type specific primer.

m+ Genomic mutant target, labelled mutant specific primer, unlabelled wild type specific primer

m− Genomic mutant target, labelled wild-type specific primer, unlabelled mutant specific primer

30 pmoles primers 30 pmoles allele specific primers used in the RC-SPA reactions

10 pmoles primers 10 pmoles allele specific primers used in the RC-SPA reactions

10 min The colour reaction proceeded for 10 minutes

It is evident that the single nucleotide specificity of the RC-SPA reactions is tolerant to at least a 3-fold range of allele specific primer concentrations.

### EXAMPLE 7

In this example, a three stage amplification was carried out to enable true interrogation of human genomic samples. First, an asymmetric polymerase chain reaction (PCR) was performed to provide a single strand amplimer containing the target fragment. This was then used as a template for a competitive ligation and the resulting circular DNA molecules amplified by a non-allele specific RCA. Finally, the amplimer from this reaction was interrogated using competitively primed RC-SPA.

Procedure

Asymmetric PCR

These reactions contained 0.1 pmole primer NATF [SEQ ID NO: 10], 50 pmole primer NATR [SEQ ID NO:11], 20 mM dNT's (Boehringer Mannheim), 5 U Taq DNA polymerase and 300-500 ng human genomic DNA and were made up to a final volume of 50 μl 1×PCR buffer+Mg (Boehringer Mannheim). The cycling conditions were as follows: 1.94° C. for 3 min, 2.30 cycles of 94° C. for 30 s, 55° C. for 30 s, 72° C. for 2 min, 3, 72° C. for 5 min.

Competitive Ligation

These reactions contained 1 pmole padlock [SEQ ID NO:3], 1 pmole SPACERW [SEQ ID NO:1], 1 pmole SPACERM [SEQ ID NO:2], 5 U ampligase DNA ligase (Epicentre Technologies) and 1 μl of asymmetric PCR reaction product in a final volume of 50 μl 1×ampligase buffer (Epicentre Technologies). The mixture was heated to 94° for 3 min then incubated at 60° C. for 20 min. The reactions were carried out in Eppendorf tubes.

Non-allele Specific RCA

The reactions were carried out in Eppendorf tubes and contained 10 mM dNTP's (Boehringer Mannheim), 10 pmoles FVComT2 [SEQ ID NO:5], 10 pmoles FVA [SEQ ID NO:4], and 1 μl of competitive ligation reaction products in a final volume of 60 μl thermopolymerase buffer (New England Biolabs). The tubes were heated at 94° C. for three minutes, then 4 U Bst polymerase large fragment (New England Biolabs) added and the wells incubated at 60° C. for 1 h.

Allele Specific Competitively Primed RC-SPA

Nunc wells coated with FVComT2 (as per Example 1) were blocked with 50 l 10 mg/ml BSA for 1 h. The blocking solution was removed from the wells and the RC-SPA reactants added. These were 10 mM dNTP's (Boehringer Mannheim), 2 pmoles FVComT2 [SEQ ID NO:5], 20 pmoles labelled FVW2 [SEQ ID NO:6] or FVM2 [SEQ ID NO:7], 20 pmoles unlabelled FVS [SEQ ID NO:8] or FV6 [SEQ ID NO:9] in a final volume of 60 μl thermopolymerase buffer (New England Biolabs). The wells were heated at 94° C. for three minutes, then 4 U Bst polymerase large fragment (New England Biolabs) added and the wells incubated at 60° C. for 10 min.

Quantitation of Immobilized Labelled Primer

This was as described in Examples 1 and 2.

Results

The A₄₀₅ readings are shown in Table 6.

TABLE 6

| | -tem | -lig | w+ | w− | m+ | m− | m+ | m− |
|---|---|---|---|---|---|---|---|---|
| 30 min | 0.051 | 0.046 | 1.405 | 0.217 | 0.546 | 0.943 | 0.428 | 0.897 |

−tem No template, labelled mutant specific primer, unlabelled wild-type specific primer

−lig Genomic mutant template, no ampligase, labelled mutant specific primer, unlabeled wild-type specific primer

w+ Genomic wild-type target, labelled wild-type specific primer, unlabelled mutant specific primer

w− Genomic wild-type target, labelled mutant specific primer, unlabelled wild type specific primer

m1+ Genomic mutant target, labelled mutant specific primer, unlabelled wild-type specific primer

m1− Genomic mutant target, labelled wild-type specific primer, unlabelled mutant specific primer

m2+ Genomic mutant target, labelled mutant specific primer, unlabelled wild-type specific primer

m2− Genomic mutant target, labelled wild-type specific primer, unlabelled mutant specific primer

30 in The colour development was for 30 min

Because the mutant genomic samples were heterozygous, it would be expected that the m1+ readings should be similar to m1− readings, and m2+ readings similar to m2− readings. However, the m1− reading was>the m1+ reading and the m2− reading>m2+ readings, indicating a greater priming activity of the wild-type specific primer. Nevertheless, the difference between the w+ and w− readings was much greater than that between the m1+ and m1−, and m2+ and m2−, thus indicating that the reaction does have specificity.

### EXAMPLE 8

An identical procedure to that outlined in Example 7 was used with the exception that the non-allele specific primer FVComT2 [SEQ ID NO:5] was not included in the solution phase during the RC-SPA. The Nunc wells are coated with this primer and the purpose of this experiment was to determine if its inclusion in the solution phase is necessary.

Results

The A₄₀₅ readings are shown in Table 8.

TABLE 8

| | -tem | -lig | w+ | w− | m+ | m− |
|---|---|---|---|---|---|---|
| 10 min | 0.053 | 0.052 | 0.591 | 0.145 | 0.427 | 0.535 |
| 25 min | 0.060 | 0.055 | 1.283 | 0.277 | 0.773 | 1.184 |

−tem: No template, labelled mutant specific primer, unlabelled wild-type specific primer

−lig Genomic mutant template, no ampligase, labelled mutant specific primer, unlabelled wild-type specific primer

w+ Genomic wild-type target, labelled wild-type specific primer, unlabelled mutant specific primer

w− Genomic wild-type target, labelled mutant-specific primer, unlabelled wild-type specific primer

m+ Genomic mutant target, labelled mutant specific primer, unlabelled wild-type specific primer

m− Genomic mutant target, labelled wild-type specific primer, unlabelled mutant specific primer

10 min The colour development was for 10 min

25 min The colour development was for 25 min

The results obtained are very similar to those from Example 8, indicating that in RC-SPA reactions that interrogate a target sequence that is previously amplified and so present in large quantities, a solution phase copy of the immobilized primer is not necessary.

### EXAMPLE 9

### Low Cycle Number Competitive Oligonucleotide Primer (COP)-PCR

The aim of this Example was to determine if low cycle number COP-PCR has the potential to achieve allele specificity for the interrogation of a SNP which exhibits little or no allele specificity using conventional allele-specific (AS)-PCR methods.

A unique base substitution in the uidA gene confers a β-glucuronidase negative phenotype allowing presumptive identification of E.coli O157:H7 (EHEC) strains. The SNP point mutation is as follows:

| | | |
|---|---|---|
| | +80 +90 | |
| E. coli | ...ATCGCGAAAACTGTGGAATTGATCAGCGTT... | [SEQ ID NO:26] |
| 0157:H7 | ...**********************G******... | |

Previously, true allele specific amplification for the SNP has not been reported, only amplification specifically for the G base substitution exclusively has been carried out to assess if tested strains are presumptive O157:H7 phenotype. Previous experiments using conventional 35 cycle allele specific amplification have shown that allele specificity for the interrogation of the uidA SNP is difficult to obtain, even when PCR reactions are optimised with respect to annealing temperature and MgCl₂ concentration.

The following methods were employed for this Example.

Samples

Samples were prepared by resuspending an overnight culture in Tris-EDTA buffer and heated at 99° C. for 20 minutes.

Low Cycle Number COP-PCR Strategy

A primer (termed the outer primer) upstream of the annealing site of the allele specific primer set was used to preamplify specific genomic sequences containing allelic variants. 50 μl PCR reaction mixes containing 2 pmol concentration of uidA5′Outer and 20 pmol concentration of uidASPA3′FAM [SEQ ID NO:22] primer (GeneWorks, Adelaide, Australia), 1 μl of genomic DNA template, 20 mM TrisCl pH 8.4, 50 mM KCl, 1.5 mM MgCl₂, 200 μM dNTPs, 0.75 unit of Platinum Tag DNA polymerase (Life Technologies). The “outer” fragment containing the SNP of interest was amplified, with cycling conditions consisting of initial denaturation (94° C., 2 minutes), 30 cycles amplification (94° C., 30 seconds), primer annealing (50, 30 seconds) and primer extension (72° C., 1 minute), followed by a final extension step (72° C., 5 minutes).

10 μl of each amplified PCR product was electrophoresed in 2% w/v agarose/TBE (45 mM Tris-borate and 1 mM EDTA, pH 8.0) for 60 mins at 100 volts to verify that an exclusive PCR product band of expect size was present. Gels were stained with ethidium bromide and visualized with a UV transilluminator (λ=302 nm). The remaining 40 μl of PCR product was purified using QIAquick (QIAGEN) PCR purification kit (Qiagen, Victoria Australia) and the dsDNA present quantified using spectrophotometry analysis.

Purified PCR product was then diluted to obtain a final concentration of 10 ng/μl and 1 μl added as template to subsequent PCR reactions set up as above using standard PCR reaction mix concentrations. Both AS-PCR and COP-PCR reactions were performed, with AS-PCR reactions consisting of 20 pmol of appropriate biotinylated primer and 20 pmol of uidASPA3′FAM [SEQ ID NO:22] fluorescein labelled common primer to each tube. COP reactions consisted of 20 pmol of uidAsmO157-B [SEQ ID NO:23] biotinylated primer was added together with 20 pmol uidAsmlEco non-biotinylated primer (i.e. allelel-B and allele 2), conversely in a separate tube 20 pmol of uidAsmlEco biotinylated primer and 20 pmol of uidAsmlO157 [SEQ ID NO:24] non-biotinylated primers were added (i.e. allele 1 and allele 2-B). Both reactions included 20 pmol of uidASPA3′FAM [SEQ ID NO:22] fluorescein labelled common primer. 10 cycles of “nested” amplification were performed consisting of initial denaturation (94° C., 2 minutes), 10 cycles amplification (94° C., 30 seconds), primer annealing (36° C., 30 seconds) and primer extension (72° C., 1 minute), followed by a final extension step (72° C., 5 minutes).

Enzyme Linked Capture and Detection Method

100 μl of a 1/200 dilution (diluted in PBS-tween, 5% w/v milk blocker solution) of PCR product was immobilized on preblocked streptavidin coated surfaces (i.e. that of microtiter plates) by shaking for 30 min at R.T. The well contents were removed and the wells were washed six times with 200 μl PBS-tween. 100 μl of anti-fluorescein-alkaline phosphatase conjugate (Roche) (at 1:25000 dilution in PBS-tween containing 5% w/v milk blocker) was added to the wells and shaken at room temperature (RT) for 30 min. Bound product was detected by the addition of 100 μl of PNPP calorimetric substrate (Sigma) and colour development monitored at 405 n using a Biomek microtiter plate-reader (Beckman).

| Primer Name | Sequences of the primers used for PCR |
|---|---|
| uidA5′ Outer | 5′-TGT AGA GCA TTA CGC TGC GAT GGA T-3′ [SEQ ID NO:21] |
| uidASPA3′FAM | 5′-FAM-TG ATG CTC CAT AAC TTC CTG-3′ [SEQ ID NO:22] |
| uidAsmO157-B | 5′-Biotin-CT GTG GAA TTG AG-3′ [SEQ ID NO:23] |
| uidAsm1O157 | 5′-CT GTG GAA TTG AG-3′ [SEQ ID NO:24] |
| uidAsmEco-B | 5′-Biotin-CT CTG GAA TTG AT [SEQ ID NO:25] |

The following results were obtained.

Competitive oligonucleotide primer PCR (COP-PCR) was used to determine what, if any, advantages such a PCR approach has on the interrogation of SNPs in comparison to AS-PCR methods.

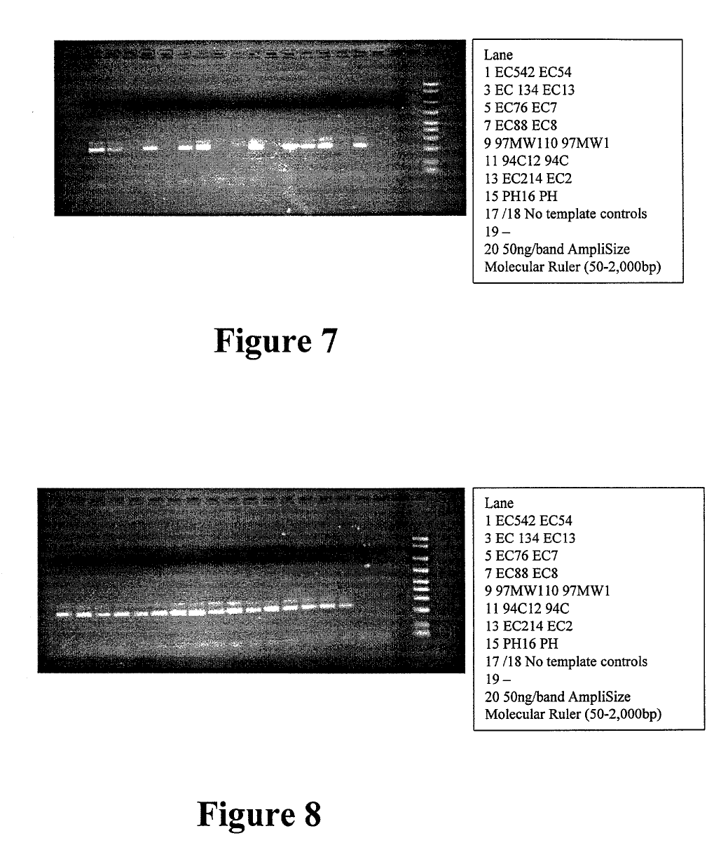

Agarose gel electrophoresis of AS PCR reactions has shown that mispriming events have occurred. At a 10 cycle stage of amplification catch up events are not yet at a stage whereby they exhibit a level of amplification which is indistinguishable from that the “true” amplimer (FIG. 7).

While SNP interrogation is possible using AS-PCR and low cycle number assessments made from gel electrophoresis results are subjective. However, from such an assessment it is observed that EC54 (Lane 1, 2) and EC 2 (Lane 13, 14) have not displayed allele specificity with bands present in both lanes. Hence, the SNP present in these sample cannot be determined.

Results from quantitative analysis of AS-PCR of the interrogation of the uida SNP have shown that two samples, EC54 and EC 2 have signal to noise ratios below 3, hence, the SNP present cannot be determined. The observed poor signal to noise ratio obtained is thought to be from these reactions undergoing mis-priming events and subsequently entering “catch up” phase at an earlier cycle number than that of the other reactions (Table 9).

Gel electrophoresis of COP-PCR products is not informative if mis-priming or allelic specificity has been obtained as both alleles primers are included in each reaction amplification always occurs. However, gel electrophoresis may be necessary to determine if equal amplification has occurred between tubes for each sample to verify that a “false” signal to noise is not being measured due to poor performance of a single PCR reaction.

Analysis of COP-PCR reaction products was carried using the enzyme linked capture and detection method as outlined above. The results are shown in FIG. 8 and Table 10.

From the quantitative data obtained from the detection assay the data indicates that all samples exhibited excellent signal to noise ratios for absorbency. Samples EC54 and EC2 have shown signal to noise ratios above 3 (5.05 and 5.09 respectively). It is noted that the average signal to noise ratio obtained using the COP-PCR is less than that of samples for AS-PCR. However, as the COP method was able to successfully interrogate the SNP for all samples, it is to be considered robust in comparison to that of AS-PCR. Sequencing of samples has confirmed that the COP-PCR has typed all but one samples correctly, however, the sequencing data from sample EC54 was inconclusive showing peaks for G and T of equal intensity. An explanation of this result may be that the sample has been contaminated with amplicon from other β-gluc positive samples hence the data for this sample must be discounted. Furthermore, the sequencing data from sample EC2 support the data from the COP-PCR in typing the SNP as a T. In comparison, AS-PCR could not interrogate the SNP reliably for EC2 with a signal to noise ratio of 1.15 whereas COP-PCR could successfully could with a signal to noise ratio of 5.09.

Upon comparison of methods, the only variable present was the type of amplification carried out, therefore, ability to successfully type all samples must be attributed to COP-PCR. Results indicate that for a given system which exhibits a high mis-priming frequency, low cycle number COP-PCR successfully produces allele specificity where conventional AS-PCR methods cannot. The results obtained support assumptions derived from theoretical modelling of such reaction systems, in that COP-PCR whilst overall showing a slightly worse signal to noise ratio than that of AS-PCR is robust with respect to catch up events derived from mis-priming events. Therefore, catch up cannot cause a significant loss of allele specificity resulting in ambiguous results and/or subsequent mistyping of allele(s) present.

TABLE 9

| | | | | | | | | | |
|---|---|---|---|---|---|---|---|---|---|
| EC54 | 0.35 | 0.2 | 1.75 | 0.0721 | 0.418 | 1.72 | 1.077 | 0.635 | 1.69 |
| EC13 | 0.048 | 1.24 | 5 | 0.073 | 0.483 | 6.6 | 0.102 | 0.724 | 7.09 |
| EC7 | 0.029 | 0.241 | 8.31 | 0.04 | 0.495 | 12.3 | 0.052 | 0.747 | 14.3 |
| EC8 | 0.351 | 0.037 | 9.4 | 0.779 | 0.049 | 15.8 | 1.168 | 0.06 | 19.4 |
| 97- | 0.031 | 0.227 | 7.3 | 0.051 | 0.5 | 9.8 | 0.069 | 0.75 | 10.8 |
| MW1 | | | | | | | | | |
| 94C | 0.027 | 0.216 | 8 | 0.041 | 0.438 | 10.6 | 0.049 | 0.652 | 13.3 |
| EC2 | 0.183 | 0.212 | 1.1 | 0.397 | 0.474 | 1.19 | 0.588 | 0.68 | 1.15 |
| PH | 0.034 | 0.194 | 5.7 | 0.046 | 0.43 | 9.3 | 0.05 | 0.632 | 12.64 |
| NO | 0.018 | 0.021 | | 0.024 | 0.024 | | 0.026 | 0.025 | |
| TEM | | | | | | | | | |

TABLE 10

| Sample | O157 10 | Eco 10 | S:N 10 m | O157 30 | Eco 30 | S:N ratio | O157 60 | Eco 60 | S:N ratio |
|---|---|---|---|---|---|---|---|---|---|
| EC54 | 0.35 | 0.2 | 1.75 | 0.0721 | 0.418 | 1.72 | 1.077 | 0.635 | 1.69 |
| EC13 | 0.048 | 0.24 | 5 | 0.073 | 0.483 | 6.6 | 0.102 | 0.724 | 7.09 |
| EC7 | 0.029 | 0.241 | 8.31 | 0.04 | 0.495 | 12.3 | 0.052 | 0.747 | 14.3 |
| EC8 | 0.351 | 0.037 | 9.4 | 0.779 | 0.049 | 15.8 | 1.168 | 0.06 | 19.4 |
| 97- | 0.031 | 0.227 | 7.3 | 0.051 | 0.5 | 9.8 | 0.069 | 0.75 | 10.8 |
| MW1 | | | | | | | | | |
| 94C | 0.027 | 0.216 | 8 | 0.041 | 0.438 | 10.6 | 0.049 | 0.652 | 13.3 |
| EC2 | 10.183 | 0.212 | 1.1 | 0.397 | 0.474 | 1.19 | 0.588 | 0.68 | 1.15 |
| PH | 0.034 | 0.194 | 5.7 | 0.046 | 0.43 | 9.3 | 0.05 | 0.632 | 12.64 |

### EXAMPLE 10

### Quantitation of Target

The method of the present invention is used to quantify target nucleic acid molecules. Two competitive primers are used in a reaction, one a perfect match (i.e. complementary) with respect to a target sequence and one comprising a single mis-match. The reaction further comprises the immobilized generic primer. The amount of target nucleic acid is determined from a standard curve wherein the amount of target is calculated by the ratio of the incorporation of the matched and mis-matched primers at the end of the amplification reaction. This method is based on the premise that the lower the amount of target, the more amplification cycles before amplification stops and the less proportional difference there is between the incorporation of matched primer and the mis-matched primer.

The use of competitive priming to quantitate target nucleic acid molecules is predicated in part on the relationship between the number of amplification cycles and the proportion of the amplimer which is mis-primed product. When amplification is exponential and 100% efficient, i.e. the amount of amplimer doubles with each cycle or unit of time, the relationship may be represented as follows:

$$\frac{b}{t o t a l} = \frac{n - 1}{4 M {e}^{\frac{( n - 2 )}{4 M}}}$$

wherein:

b=homoduplex DNA that is the opposite allele as the target (i.e. amount of mis-primed product)

total=amount of amplimer

n=PCR cycle number

M=mis-priming frequency written as a whole number

e=the exponential constant (2.718281828).

This function may be graphically plotted and shows that in a competitive priming reaction, the more cycles of amplification, the greater the proportion of mis-primed product in comparison to total amplimer.

When there is an unknown amount of target, the number of amplification cycles will be a function of the amount of target; the more target, the fewer the cycles required before the reaction runs out of monomers as the maximum amount of amplimer that can be synthesized is reached. Therefore, the

$$( \frac{b}{t o t a l} )$$

figure is a function of the amount of target.

Consequently, if

$$\frac{b}{t o t a l}$$

is measured, then the amount of target can be calculated.

### EXAMPLE 11

The instant assay enables the identification and/or discrimination between nucleotide repeat number polymorphism such as microsatellite repeat number alleles when the number of repeats contained within an amplified DNA fragment is above or below a pre-defined threshold value.

In one particular aspect of the present invention, a pair of solid phase oligonucleotides are used. These may be in separate wells or on separate array spots. The target nucleic acid molecule is then subjected to non-competitive primer extension. The extension is at or near the nucleotide length polymorphism or microsatellite. Consequently, one primer is capable of inducing extension whereas the other primer is not. By using incorporation of a label, the extension or lack of extension is thereby detectable. This concept is shown diagrammatically in FIG. 7. In this depiction, a target DNA molecule comprises a repeat region indicated by a wavy line. The target DNA is subjected to interrogation using a pair of primers where one primer is capable of priming and label incorporation and the other primer is not, due to its length.

The nucleotide length polymorphism or microsatellite DNA may be used as an indicator of the presence or absence of a particular disease condition or a propensity for development of said disease condition such as but not limited to a neurodegenerative disorder (i.e. Huntington's disease).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

1. Saiki et al, Science 230: 1350-1354, 1985.

2. Landegren et al, Science 241: 1077-1080, 1988.

3. Kwoh et al, Proceedings of the National Academy of Sciences, USA 86: 1173-1177, 1989.

4. Lizardi, et al, Bio/technology 6: 1197-1202, 1988.

5. Guatelli, et al, Proceedings of the National Academy of Sciences, USA 87: 1874-1878, 1990.

6. Kievitis T et al, Journal of Virological Methods 35: 273-286, 1991.

7. Walker et al, Nucleic Acids Research 20: 1691-1696, 1992.

8. Lizardi et al, Nature Genetics 19: 225-232, 1998.

9. Zhang et al, Gene 211: 277-285, 1998.

10. Nillson et al., Science 265: 2085-2088, 1994.

11. Cerrone et al., Infect. Immun. 59: 79-90, 1991.

12. Kikuta et al., Infect. Immun. 59: 4665-4669, 1991.

13. Bertina et al., Nature 369: 64-67, 1994

1. A method for detecting a nucleic acid molecule having a particular nucleotide sequence immobilized to a solid support via hybridization of said nucleic acid molecule to a primer anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is for a time and under conditions sufficient for the nucleic acid primer which is complementary to the target sequence to hybridize to said target sequence with greater efficiency and/or specificity compared to the nucleic acid primer which contains a mis-match and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

2. A method according to claim 1 wherein a solution phase primer is allele specific and is capable of extending from its 3′ terminus to form an extension product complementary to the strand to which the primer has hybridized.

3. A method for detecting a nucleic acid molecule having a particular nucleotide sequence immobilized to a solid support via hybridization of said nucleic acid molecule to a primer anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

4. A method according to claim 1 or 2 or 3 wherein the competitive priming step occurs after amplification with a generic primer.

5. A method for detecting a nucleic acid molecule having a particular nucleotide sequence anchored to a solid support via hybridization of said nucleic acid molecule to a primer anchored to said solid support, said method comprising subjecting said nucleic acid molecule to amplification using at least two solution phase primers and anchoring said amplified nucleic acid molecule to the solid support, contacting said anchored nucleic acid molecule with at least two other solution phase nucleic acid primers wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence with greater efficiency and/or specificity compared to the nucleic acid primer which contains a mis-match and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

6. A method according to claim 5 wherein the solution phase primer is allele specific and is capable of extending from its 3′ terminus to form an extension product complementary to the strand to which the primer has hybridized.

7. A method for detecting a nucleic acid molecule having a particular nucleotide sequence anchored to a solid support via hybridization of said nucleic acid molecule to a primer anchored to said solid support, said method comprising subjecting said nucleic acid molecule to amplification using at least two solution phase primers and anchoring said amplified nucleic acid molecule to the solid support, contacting said anchored nucleic acid molecule with at least two other solution phase nucleic acid primers wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

8. A method for detecting a nucleic acid molecule having a particular nucleotide sequence anchored to a solid support via hybridization of said nucleic acid molecule to a primer anchored to said solid support, said method comprising subjecting said nucleic acid molecule to amplification using at least two solution phase primers having a high Tₘ and wherein the amplification conditions are such that said solution phase primers are active and anchoring said amplified nucleic acid molecule to the solid support, contacting said anchored nucleic acid molecule with at least two other solution phase nucleic acid primers wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said latter two primers have a Tₘ lower than the temperature used in said first amplification such that during the first amplification, the second set of primers is inactive but during said second amplification, said second set of primers is active, wherein said contact is under conditions sufficient for the nucleic acid primer which is complementary to the target sequence to hybridize to said target sequence greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

9. A method for detecting a nucleic acid molecule having a particular nucleotide sequence anchored to a solid support via hybridization of said nucleic acid molecule to a primer anchored to said solid support, said method comprising subjecting said nucleic acid molecule to amplification using a solution phase primer having a high Tₘ and wherein the amplification conditions are such that said solution phase primers are active and anchoring said amplified nucleic acid molecule to the solid support, contacting said anchored nucleic acid molecule with at least two other solution phase nucleic acid primers each having a low Tₘ wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said latter two primers have a Tₘ lower than the temperature used in said first amplification such that during the first amplification, the second set of primers is inactive but during said second amplification, said second set of primers is active, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

10. A method according to claim 8 or 9 wherein the immobilized primer has a high Tₘ.

11. A method according to claim 8 or 9 wherein the immobilized primer has a low Tₘ.

12. A solid support comprising an array of immobilized primers wherein each of the primers may comprise the identical nucleotide sequence or one or more may differ from each other by at least one nucleotide and wherein the array is used to detect a nucleic acid molecule having a particular nucleotide sequence immobilized to a solid support via hybridization of said nucleic acid molecule to said one or more primers anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence with greater efficiency and/or specificity compared to the nucleic acid primer which contains a mis-match and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

13. A solid support according to claim 12 wherein a solution phase primer is allele specific and is capable of extending from its 3′ terminus to form an extension product complementary to the strand to which the primer has hybridized.

14. A solid support comprising an array of immobilized primers wherein each of the primers may comprise the identical nucleotide sequence or one or more may differ from each other by at least one nucleotide and wherein the array is used to detect a nucleic acid molecule having a particular nucleotide sequence immobilized to a solid support via hybridization of said nucleic acid molecule to said one or more primers anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence but at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

15. A nucleic acid molecule having a particular nucleotide sequence anchored to a solid support via hybridization of said nucleic acid molecule to a primer anchored to said solid support, said method comprising subjecting said nucleic acid molecule to amplification using at least two solution phase primers having a high Tₘ and wherein the amplification conditions are such that said solution phase primers are active and anchoring said amplified nucleic acid molecule to the solid support, contacting said anchored nucleic acid molecule with at least two other solution phase nucleic acid primers wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said latter two primers have a Tₘ lower than the temperature used in said first amplification such that during the first amplification, the second set of primers is inactive but during said second amplification, said second set of primers is active, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

16. A nucleic acid molecule having a particular nucleotide sequence anchored to a solid support via hybridization of said nucleic acid molecule to a primer anchored to said solid support, said method comprising subjecting said nucleic acid molecule to amplification using a solution phase primer having a high Tₘ at least two solution phase primers having a high Tₘ and wherein the amplification conditions are such that said solution phase primers are active and anchoring said amplified nucleic acid molecule to the solid support, contacting said anchored nucleic acid molecule with at least two other solution phase nucleic acid primers each having a low Tₘ wherein the nucleotide sequence of at least one of the primers is complementary to a target nucleotide sequence within or on the anchored nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said latter two primers have a Tₘ lower than the temperature used in said first amplification such that during the first amplification, the second set of primers is inactive but during said second amplification, said second set of primers is active, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the anchored nucleic acid molecule.

17. A method for discriminating between nucleotide repeat number polymorphism on a nucleic acid molecule immobilized to a solid support via hybridization of said nucleic acid molecule to a primer anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is for a time and under conditions sufficient for the nucleic acid primer which is complementary to the target sequence to hybridize to said target sequence with greater efficiency and/or specificity compared to the nucleic acid primer which contains a mis-match and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

18. A method according to claim 17 wherein a solution phase primer is allele specific and is capable of extending from its 3′ terminus to form an extension product complementary to the strand to which the primer has hybridized.

19. A method for discriminating between nucleotide repeat number polymorphism on a nucleic acid molecule immobilized to a solid support via hybridization of said nucleic acid molecule to a primer anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primer which is complementary to the target sequence to hybridize to said target sequence and to extend from its 3′ terminus to form an extension product complementary to the strand to which the primer has hybridized with greater efficiency and/or specificity compared to a mis-matched primer and then detecting the relative presence of a signal wherein the relative presence of said signal is indicative of which primer has hybridized to the target sequence depending on which primer has been labelled and the relative presence of a signal is indicative of the identity of the immobilized nucleic acid molecule.

20. A method according to claim 17 or 18 or 19 wherein the nucleotide repeat number polymorphism is microsatellite DNA.

21. A method according to claim 17 or 18 or 19 or 20 to detect a neurodegenerative disease.

22. A method according to claim 21 wherein the neurodegenerative disease is Huntington's disease.

23. A method for detecting a nucleotide repeat number polymorphism in a target nucleic acid molecule, said method comprising subjecting said target nucleic acid molecule to interrogation by a pair of immobilized primers wherein said primers are immobilized in separate reaction vessels or separate spot arrays and wherein one primer is capable of priming a particular nucleotide length polymorphism whereas the other primer is unable to induce priming of said nucleotide length polymorphism wherein at least one primer is labelled with a reporter molecule capable of providing an identifiable signal wherein the relative presence or absence of said signal is indicative of the presence or absence of said nucleotide length polymorphism.

24. A method according to claim 23 wherein the nucleotide repeat number polymorphism is microsatellite DNA.

25. A method according to 23 to detect a neurodegenerative disease.

26. A method according to claim 23 wherein the neurodegenerative disease is Huntington's disease.

27. A method for quantitating a nucleic acid molecule immobilized to a solid support via hybridization of said nucleic acid molecule to a primer anchored to said solid support, said method comprising contacting said immobilized nucleic acid molecule with at least two solution phase nucleic acid primers wherein the nucleotide sequence of at least one of the primers is complementary to a target nucleotide sequence within or on the immobilized nucleic acid molecule and wherein the nucleotide sequence of at least another primer differs from said target nucleotide sequence by at least one nucleotide mis-match and wherein at least one of said at least two primers is labelled with a reporter molecule capable of providing an identifiable signal, wherein said contact is under conditions sufficient for the nucleic acid primers to amplify said target sequence to completion of an amplification reaction wherein the amount of target nucleic acid molecule is proportional to the number of amplification cycles such that the amount of target nucleic acid molecule is determined from the ratio of incorporation of complementary and mis-match primers at the end of the amplification reaction.


SEQUENCE LISTING (26 sequences)

SEQ ID NO: 1 - DNA, 18 bp, artificial sequence
  misc_feature ()..(): SPACERW
cagatccctggacaggcg

SEQ ID NO: 2 - DNA, 18 bp, artificial sequence
  misc_feature ()..(): SPACERM
cagatccctggacaggca

SEQ ID NO: 3 - DNA, 83 bp, artificial sequence
  misc_feature ()..(): PADLOCK
aggaatacaggtattttgtccttgcgcggtgagctatatggggactatgaatttctaataggactacttctaatctgtaagag

SEQ ID NO: 4 - DNA, 20 bp, artificial sequence
  misc_feature ()..(): FVA
aatctgtaagagcagatccc

SEQ ID NO: 5 - DNA, 29 bp, artificial sequence
  misc_feature ()..(): FVComT2
ttttttttttgtccccatatagctcaccg

SEQ ID NO: 6 - DNA, 18 bp, artificial sequence
  misc_feature ()..(): FVW2
cagatccctggacagacg

SEQ ID NO: 7 - DNA, 18 bp, artificial sequence
  misc_feature ()..(): FVM2
cagatccctggacagaca

SEQ ID NO: 8 - DNA, 16 bp, artificial sequence
  misc_feature ()..(): FV5
gatccctggacagacg

SEQ ID NO: 9 - DNA, 16 bp, artificial sequence
  misc_feature ()..(): FV6
gatccctggacagaca

SEQ ID NO: 10 - DNA, 22 bp, artificial sequence
  misc_feature ()..(): NATF
tgcccagtgcttaacaagacca

SEQ ID NO: 11 - DNA, 20 bp, artificial sequence
  misc_feature ()..(): NATR
tgttatcacactggtgctaa

SEQ ID NO: 12 - DNA, 67 bp, artificial sequence
  misc_feature ()..(): Chlamydia-specific padlock
gcaggtaaagaaggcgccgcggtgagctatatggggactatgaatttgctccattaaagcaaattgc

SEQ ID NO: 13 - DNA, 49 bp, artificial sequence
  misc_feature ()..(): Chlamydia pneumoniae-derived target
atagcgccttctttacctgcgttacttcgaatttgctttaatggagctg

SEQ ID NO: 14 - DNA, 7 bp, artificial sequence
  misc_feature ()..(): Chlamydia pneumoniae-specific spacer
aagtaac

SEQ ID NO: 15 - DNA, 49 bp, artificial sequence
  misc_feature ()..(): Chlamydia trachomatis-derived target
atagcaccttcttttcctgcgtttgcacgaatttgtttcaaaggagcgg

SEQ ID NO: 16 - DNA, 7 bp, artificial sequence
  misc_feature ()..(): Chlamydia trachomatis-specific spacer
tgcaaac

SEQ ID NO: 17 - DNA, 20 bp, artificial sequence
  misc_feature ()..(): Chlamydia pneumoniae-specific amplification primer
ccattaaagcaaattgcaag

SEQ ID NO: 18 - DNA, 21 bp, artificial sequence
  misc_feature ()..(): Chlamydia trachomatis-specific amplification primer
ccattaaagcaaattgctgca

SEQ ID NO: 19 - DNA, 18 bp, artificial sequence
  misc_feature ()..(): Wild-type FVComT
cagatccctggacaggcg

SEQ ID NO: 20 - DNA, 18 bp, artificial sequence
  misc_feature ()..(): Mutant FVComT
cagatccctggacaggca

SEQ ID NO: 21 - DNA, 25 bp, artificial sequence
  misc_feature ()..(): UidA3′ outer
tgtagagcattacgctgcgatggat

SEQ ID NO: 22 - DNA, 20 bp, artificial sequence
  misc_feature ()..(): UidASPA3′FAM
tgatgctccataacttcctg

SEQ ID NO: 23 - DNA, 13 bp, artificial sequence
  misc_feature ()..(): UidAsm0157-B
ctgtggaattgag

SEQ ID NO: 24 - DNA, 13 bp, artificial sequence
  misc_feature ()..(): UidAsm1O157
ctgtggaattgag

SEQ ID NO: 25 - DNA, 13 bp, artificial sequence
  misc_feature ()..(): UidAsmEco-B
ctctggaattgat

SEQ ID NO: 26 - DNA, 30 bp, artificial sequence
  misc_feature ()..(): E. coli O157 H7
atcgcgaaaactgtggaattgatcagcgtt